SEQUENCE LISTING (43 sequences)

SEQ ID NO: 1 - DNA, 3996 bp, Homo sapiens
  misc_feature (1)..(3996)
agtttctgccaggagctaatatggcttccttagttacaccgttctctctcttcacctaatcagcgaccttactttcccagaccagactgtcgagcaggagctaagactccttttcccctctgctgaccgccactacaggagcggttgaagccagacgaccaccttgtggagttaaactccgtaaccagggagcaccacttccgctgacgtcattacggcgacacgtggatccaagatggcgacggcgatggattggttgccgtggtctttactgcttttctccctgatgtgtgaaacaagcgccttctatgtgcctggggtcgcgcctatcaacttccaccagaacgatcccgtagaaatcaaggctgtgaagctcaccagctctcgaacccagctaccttatgaatactattcactgcccttctgccagcccagcaagataacctacaaggcagagaatctgggagaggtgctgagaggggaccggattgtcaacacccctttccaggttctcatgaacagcgagaagaagtgtgaagttctgtgcagccagtccaacaagccagtgaccctgacagtggagcagagccgactcgtggccgagcggatcacagaagactactacgtccacctcattgctgacaacctgcctgtggccacccggctggagctctactccaaccgagacagcgatgacaagaagaaggaaaaagatgtgcagtttgaacacggctaccggctcggcttcacagatgtcaacaagatctacctgcacaaccacctctcattcatcctttactatcatcgggaggacatggaagaggaccaggagcacacgtaccgtgtcgtccgcttcgaggtgattccccagagcatcaggctggaggacctcaaagcagatgagaagagttcgtgcactctgcctgagggtaccaactcctcgccccaagaaattgaccccaccaaggagaatcagctgtacttcacctactctgtccactgggaggaaagtgatatcaaatgggcctctcgctgggacacttacctgaccatgagtgacgtccagatccactggttttctatcattaactccgttgttgtggtcttcttcctgtcaggtatcctgagcatgattatcattcggaccctccggaaggacattgccaactacaacaaggaggatgacattgaagacaccatggaggagtctgggtggaagttggtgcacggcgacgtcttcaggcccccccagtaccccatgatcctcagctccctgctgggctcaggcattcagctgttctgtatgatcctcatcgtcatctttgtagccatgcttgggatgctgtcgccctccagccggggagctctcatgaccacagcctgcttcctcttcatgttcatgggggtgtttggcggattttctgctggccgtctgtaccgcactttaaaaggccatcggtggaagaaaggagccttctgtacggcaactctgtaccctggtgtggtttttggcatctgcttcgtattgaattgcttcatttggggaaagcactcatcaggagcggtgccctttcccaccatggtggctctgctgtgcatgtggttcgggatctccctgcccctcgtctacttgggctactacttcggcttccgaaagcagccatatgacaaccctgtgcgcaccaaccagattccccggcagatccccgagcagcggtggtacatgaaccgatttgtgggcatcctcatggctgggatcttgcccttcggcgccatgttcatcgagctcttcttcatcttcagtgctatctgggagaatcagttctattacctctttggcttcctgttccttgttttcatcatcctggtggtatcctgttcacaaatcagcatcgtcatggtgtacttccagctgtgtgcagaggattaccgctggtggtggagaaatttcctagtctccgggggctctgcattctacgtcctggtttatgccatcttttatttcgttaacaagctggacatcgtggagttcatcccctctctcctctactttggctacacggccctcatggtcttgtccttctggctgctaacgggtaccatcggcttctatgcagcctacatgtttgttcgcaagatctatgctgctgtgaagatagactgattggagtggaccacggccaagcttgctccgtcctcggacaggaagccaccctgcgtgggggactgcaggcacgcaaaataaaataactcctgctcgtttggaatgtaactcctggcacagtgttcctggatcctggggctgcgtggggggcgggagggcctgtagataatcttgcgtttttcgtcatcttattccagttctgtgggggatgagtttttttgtgggttgctttttcttcagtgctaagaaagttccctccaacaggaactctctgacctgtttattcaggtgtatttctggtttggatttttttttccttctttgttttaacaaatggatccaggatggataaatccaccgagataagggttttggtcactgtctccacctcagttcctcagggctgttggccaccctatgactaactggaagaggacacgccagagcttcagtgaggtttccgagcctctccctgcccatcctcaccactgaggccacgacaaagcacagctccagctcggacagcaccctcagtgccagccagcctctgccagacctctctttccctcttctccccagcctcctccagggctgcccaaggcagggtttccagccaggcctcggggtcatcttttcaccaggagcaaacccaagtcttagttgctacaagaaaatcccctggaagtactgggggccaggttccccagacagcaggaattgcccctgttcagagcagccggagtttgctggaccacaaggaagaagagaagagacttgcagtgaactgtttttgtgccaagaaaccctggacctggggccaagtatttcccaagccaagcatccacttgtctgtgtctgggaagggatggccaaggccgctagggtccttacccctcaggatcactccccagccctttcctcaggaggtaccgctctccaaggtgtgctagcagtgggccctgcccaacttcaggcagaacagggaggcccagagattacagatcccctcctgtaagtggccaggcattctctccctgccctctctggcctctggggtcatactcacttctttagccagccccatcccctccaccccacacctgagttcttgcctcctccttttggggacacccaaaacactgcttgtgagaaggaagatggaaggtaagttctgtcgttctttccccaatccccaggaatggacaagaagccaacttagaaagaagggtctcacgtggctggcctggctcctccgtagacccctgttcttttcaacctctgcccacccgtgcatgtcatcacaaacatttgctcttaagttacaagagaccacatccacccagggattagggttcaagtagcagctgctaacccttgcaccagcccttgtgggactcccaacacaagacaaagctcaggatgctggtgatgctaggaagatgtccctcccctcactgccccacattctcccagtggctctaccagcctcacccatcaaaccagtgaatttctcaatcttgcctcacagtgactgcagcgccaagcggcatccaccaagcatcaagttggagaaaagggaacccaagcagtagagagcgatattggagtcttttgttcattcaaatcttggatttttttttttccctaagagattctctttttagggggaatgggaaacggacacctcataaagggttcaaagatcatcaatttttctgactttttaaatcattatcattattatttttaattaaaaaaatgcctgtatgcctttttttggtcggattgtaaataaatataccattgtcctactgaaaaaaaaaaaaaaaaaa

SEQ ID NO: 2 - protein, 642 aa, Homo sapiens
  MISC_FEATURE (1)..(642): TM9SF4/TUCAP-1 protein
MATAMDWLPWSLLLFSLMCETSAFYVPGVAPINFHQNDPVEIKAVKLTSSRTQLPYEYYSLPFCQPSKITYKAENLGEVLRGDRIVNTPFQVLMNSEKKCEVLCSQSNKPVTLTVEQSRLVAERITEDYYVHLIADNLPVATRLELYSNRDSDDKKKEKDVQFEHGYRLGFTDVNKIYLHNHLSFILYYHREDMEEDQEHTYRVVRFEVIPQSIRLEDLKADEKSSCTLPEGTNSSPQEIDPTKENQLYFTYSVHWEESDIKWASRWDTYLTMSDVQIHWFSIINSVVVVFFLSGILSMIIIRTLRKDIANYNKEDDIEDTMEESGWKLVHGDVFRPPQYPMILSSLLGSGIQLFCMILIVIFVAMLGMLSPSSRGALMTTACFLFMFMGVFGGFSAGRLYRTLKGHRWKKGAFCTATLYPGVVFGICFVLNCFIWGKHSSGAVPFPTMVALLCMWFGISLPLVYLGYYFGFRKQPYDNPVRTNQIPRQIPEQRWYMNRFVGILMAGILPFGAMFIELFFIFSAIWENQFYYLFGFLFLVFIILVVSCSQISIVMVYFQLCAEDYRWWWRNFLVSGGSAFYVLVYAIFYFVNKLDIVEFIPSLLYFGYTALMVLSFWLLTGTIGFYAAYMFVRKIYAAVKID

SEQ ID NO: 3 - DNA, 2391 bp, Homo sapiens
  misc_feature (1)..(2391): TM9SF2 encoding nucleic acid
cgcaaccggaactagccttctgggggccggcttggtttatctctggcggccttgtagtcgtctccgagactccccacccctccttccctcttgaccccctaggtttgattgccctttccccgaaacaactatcatgagcgcgaggctgccggtgttgtctccacctcggtggccgcggctgttgctgctgtcgctgctcctgctgggggcggttcctggcccgcgccggagcggcgctttctacctgcccggcctggcgcccgtcaacttctgcgacgaagaaaaaaagagcgacgagtgcaaggccgaaatagaactatttgtgaacagacttgattcagtggaatcagttcttccttatgaatacacagcgtttgatttttgccaagcatcagaaggaaagcgcccatctgaaaatcttggtcaggtactattcggggaaagaattgaaccttcaccatataagtttacgtttaataagaaggagacctgtaagcttgtttgtacaaaaacataccatacagagaaagctgaagacaaacaaaagttagaattcttgaaaaaaagcatgttattgaattatcaacatcactggattgtggataatatgcctgtaacgtggtgttacgatgttgaagatggtcagaggttctgtaatcctggatttcctattggctgttacattacagataaaggccatgcaaaagatgcctgtgttattagttcagatttccatgaaagagatacattttacatcttcaaccatgttgacatcaaaatatactatcatgttgttgaaactgggtccatgggagcaagattagtggctgctaaacttgaaccgaaaagcttcaaacatacccatatagataaaccagactgctcagggccccccatggacataagtaacaaggcttctggggagataaaaattgcctatacttactctgttagcttcgaggaagatgataagatcagatgggcgtctagatgggactatattctggagtctatgcctcatacccacattcagtggtttagcattatgaattccctggtcattgttctcttcttatctggaatggtagctatgattatgttacggacactgcacaaagatattgctagatataatcagatggactctacggaagatgcccaggaagaatttggctggaaacttgttcatggtgatatattccgtcctccaagaaaagggatgctgctatcagtctttctaggatccgggacacagattttaattatgacctttgtgactctatttttcgcttgcctgggatttttgtcacctgccaaccgaggagcgctgatgacgtgtgctgtggtcctgtgggtgctgctgggcacccctgcaggctatgttgctgccagattctataagtcctttggaggtgagaagtggaaaacaaatgttttattaacatcatttctttgtcctgggattgtatttgctgacttctttataatgaatctgatcctctggggagaaggatcttcagcagctattccttttgggacactggttgccatattggccctttggttctgcatatctgtgcctctgacgtttattggtgcatactttggttttaagaagaatgccattgaacacccagttcgaaccaatcagattccacgtcagattcctgaacagtcgttctacacgaagcccttgcctggtattatcatgggagggattttgccctttggctgcatctttatacaacttttcttcattctgaatagtatttggtcacaccagatgtattacatgtttggcttcctatttctggtgtttatcattttggttattacctgttctgaagcaactatacttctttgctatttccacctatgtgcagaggattatcattggcaatggcgttcattccttacgagtggctttactgcagtttatttcttaatctatgcagtacactacttcttttcaaaactgcagatcacgggaacagcaagcacaattctgtactttggttataccatgataatggttttgatcttctttctttttacaggaacaattggcttctttgcatgcttttggtttgttaccaaaatatacagtgtggtgaaggttgactgaagaagtccagtgtgtccagttaaaacagaaataaattaaactcttcatcaacaaagacctgtttttgtgactgccttgagttttatcagaattattggcctagtaatccttcagaaacaccgtaattctaaataaacctcttcccatacacctttcccccataagatctgtcttcaacactataaagcatttgtattgtgatttgattaagtatatatttggttgttctcaatgaagagcaaatttaaatattatgtgcatttgaa

SEQ ID NO: 4 - protein, 663 aa, Homo sapiens
  MISC_FEATURE (1)..(663): TM9SF2 protein
MSARLPVLSPPRWPRLLLLSLLLLGAVPGPRRSGAFYLPGLAPVNFCDEEKKSDECKAEIELFVNRLDSVESVLPYEYTAFDFCQASEGKRPSENLGQVLFGERIEPSPYKFTFNKKETCKLVCTKTYHTEKAEDKQKLEFLKKSMLLNYQHHWIVDNMPVTWCYDVEDGQRFCNPGFPIGCYITDKGHAKDACVISSDFHERDTFYIFNHVDIKIYYHVVETGSMGARLVAAKLEPKSFKHTHIDKPDCSGPPMDISNKASGEIKIAYTYSVSFEEDDKIRWASRWDYILESMPHTHIQWFSIMNSLVIVLFLSGMVAMIMLRTLHKDIARYNQMDSTEDAQEEFGWKLVHGDIFRPPRKGMLLSVFLGSGTQILIMTFVTLFFACLGFLSPANRGALMTCAVVLWVLLGTPAGYVAARFYKSFGGEKWKTNVLLTSFLCPGIVFADFFIMNLILWGEGSSAAIPFGTLVAILALWFCISVPLTFIGAYFGFKKNAIEHPVRTNQIPRQIPEQSFYTKPLPGIIMGGILPFGCIFIQLFFILNSIWSHQMYYMFGFLFLVFIILVITCSEATILLCYFHLCAEDYHWQWRSFLTSGFTAVYFLIYAVHYFFSKLQITGTASTILYFGYTMIMVLIFFLFTGTIGFFACFWFVTKIYSVVKVD

SEQ ID NO: 5 - DNA, 6140 bp, Homo sapiens
  misc_feature (1)..(6140): TM9SF3 encoding sequence
gaggaagaggctgaggaggcgcggggggcgggggaggctcaggagcgggcggtgacggcgacggcggcggcagaggaggcagcggctgggccgggccccgtgcgtctgtccgcgccccgtggatgcgaatcggccgcggcggaggcggcggcggcggaggaggcggcggcgggaggaggagtcggtgagccggctccgggccggaggggcgcggaggatgaggccgctgcctggcgctcttggcgtggcggcggccgccgcgctgtggctgctgctgctgctgctgccccggacccgggcggacgagcacgaacacacgtatcaagataaagaggaagttgtcttatggatgaatactgttgggccctaccataatcgtcaagaaacatataagtacttttcacttccattctgtgtggggtcaaaaaaaagtatcagtcattaccatgaaactctgggagaagcacttcaaggggttgaattggaatttagtggtctggatattaaatttaaagatgatgtgatgccagccacttactgtgaaattgatttagataaagaaaagagagatgcatttgtatatgccataaaaaatcattactggtaccagatgtacatagatgatttaccaatatggggtattgttggtgaggctgatgaaaatggagaagattactatctttggacctataaaaaacttgaaataggttttaatggaaatcgaattgttgatgttaatctaactagtgaaggaaaggtgaaactggttccaaatactaaaatccagatgtcatattcagtaaaatggaaaaagtcagatgtgaaatttgaagatcgatttgacaaatatcttgatccgtccttttttcaacatcggattcattggttttcaattttcaactccttcatgatggtgatcttcttggtgggcttagtttcaatgattttaatgagaacattaagaaaagattatgctcggtacagtaaagaggaagaaatggatgatatggatagagacctaggagatgaatatggatggaaacaggtgcatggagatgtatttagaccatcaagtcacccactgatattttcctctctgattggttctggatgtcagatatttgctgtgtctctcatcgttattattgttgcaatgatagaagatttatatactgagaggggatcaatgctcagtacagccatatttgtctatgctgctacgtctccagtgaatggttattttggaggaagtctgtatgctagacaaggaggaaggagatggataaagcagatgtttattggggcattccttatcccagctatggtgtgtggcactgccttcttcatcaatttcatagccatttattaccatgcttcaagagccattccttttggaacaatggtggccgtttgttgcatctgtttttttgttattcttcctctaaatcttgttggtacaatacttggccgaaatctgtcaggtcagcccaactttccttgtcgtgtcaatgctgtgcctcgtcctataccggagaaaaaatggttcatggagcctgcggttattgtttgcctgggtggaattttaccttttggttcaatctttattgaaatgtatttcatcttcacgtctttctgggcatataagatctattatgtctatggcttcatgatgctggtgctggttatcctgtgcattgtgactgtctgtgtgactattgtgtgcacatattttctactaaatgcagaagattaccggtggcaatggacaagttttctctctgctgcatcaactgcaatctatgtttacatgtattccttttactactattttttcaaaacaaagatgtatggcttatttcaaacatcattttactttggatatatggcggtatttagcacagccttggggataatgtgtggagcgattggttacatgggaacaagtgcctttgtccgaaaaatctatactaatgtgaaaattgactagagacccaagaaaacctggaactttggatcaatttctttttcataggggtggaacttgcacagcaaaaacaaacaaacgcaagaagagatttgggctttaacacactgggtactttgtgggtctctctttcgtcggtggcttaaagtaacatctatttccattgatcctaggttcttcctgactgctttctccaactgttcacagcaaatgcttggattttatgcagtaggcattactacagtacatggctaatcttcccaaaaactagctcattaaagatgaaatagaccagctctcttcagtgaagaggacaaatagtttatttaaagcatttgttccaataaaataaatagagggaaacttggatgctaaaattacatgaataggaatcttcctggcacttagtgtttctatgttattgaaaaatgatgttccagaaagattacttttttcctcttatttttactgccattgtcgacctattgtgggacatttttatatattgaatctgggttcttttttgactttttttttttcccaatccaacagcatccttttttttaaaagagagaattagaaaatattaaatcctgcatgtaatatatctgctgtcatcttagttggaccaacttcccatttatttatcttaaaactatacagttacatcttaattccatccaaagaagatacagtttgaagacagaagtgtactctctacaatgcaatttactgtacagttagaaagcaaagtgttaaatggagaagatacttgtttttattaaacattttgagatttagataaactacattttaactgaatgtctaaagtgattatcttttttccccccaagttagtcttaaatcttttgggtttgaatgaaggttttacataagaaattattaaaaacaaggggggtgggtaataaatgtatataacattaaataatgtaacgtaggtgtagattcccaaatgcatttggatgtacagatcgactacagagtacttttttcttatgatgattggtgtagaaatgtgtgatttgggtgggcttttacatcttgcctaccattgcatgaaacattggggtttcttcaaaatgtgtgtgtcatacttcttttgggaggggggttgttttcttctgtttattttctgagactcctacaggagccaaatttgtaatttagagacacttaattttgttaatcctgtctgggacacttaagtaacatctaaagcattattgctttagaatgttcaaataaaatttcctgaccaaattgttttgtggaaatagatgtgtttgcaatttgaagatatctttctgtccagaaggcaaaattaccgaatgccatttttaaaagtatgctataaactatgctactctcatacaggggacccgtattttaaaatctccagacttgcttacatctagattatccagcacaatcataaagtgaatgacaaaccctttgaatgaaattgtggcacaaaatctgttcaggttggtgtaccgtgtaaagtggggatggggtaaaagtggttaacgtactgttggatcaacaaataaaggttacagttttgtaagagaagtgatttgaatacatttttctggaactattcataatatgaagttttcctagaaccactgagtttctagtttaatagtttgctatgcaaatgaccacctaaaacaatactttatattgttatttttagaaagactcaaaacacctgtatttaaaccttaatatgaaaatcatgcaattaatagttacacaagatgttttcattacaaaatatgtacctatctattgatggactctacatcctatattgtgacatgtaagtcctttaaaaggtgaaaagtatgatttcttaccacttaagtatgattgatatgatccaacaaatttgatcagaagctgtaggtaaatcctcttctgaagccaaaatggtatattaaatataatttattggtacttccattttctcttccttcttacttgcctttaagatcttataaaaaagaaactaaaagttaatatttagttgcctatattatgtaaccttttaactatatataaagtacttttttggtttctttctcaccacttttattcaaaagtacttttaacataccaatacatagtctgtctgatgggagtataaattggacagtaaggttttgtcttaataaaatgaaatttgtttctcatgatatgaatcttgcaggtaagatgtagggtttattgaaaatgtgtgggttaaatgctttcaggtacaccaattctttctactaaattgagctctatttgaagttctttggaatctgtggtgaaaaataattttctgatttccaaatacattaagagcattaaatgaatattaatcacctttaaagtcttttagaaaaggacttgtattggtttttggctgcatagaggggttgaataagtgtatgtatgtgtgtgcgtgtgtgtgtgtcttcttaaagaagatgtaattcacaaatagtttagctccctagcgctcagttgtagaatagaaaatagaacattattcaagttaattgaaaggtgaggtttttatacccccactaatgctgtgtatctgtctttcgtttgttaacattatttgcttaatttctttcaactcacactttggataatactatcaaaaactaaggctaaacattccttgtgtatctttaagcatgcttctcctgaaatttaactacattagtagttgacatttgtatacatatatcctaatacaagagtaggataaggtggaaatgtaatggcctgagggatggtgaagcattcttttagtatttttcatcatgttgggctcctagattgtactggggttgcccataaatcaaaccccatactcttagaattcattatattatggtgatatccgaacctagtgaatggtatgcttgggtgttttccattgagagtggatggacctctttataaagttggttgctgcaaaatccagttcttccaaaagccactttatttagggtttattcacaagtcatatccattttggtacagtgtttgtttcctaatatttattaaccaccttataccaaatgtcttgcaaagaaatgttattaaaaccttgaatttttacaaatgtaaaaaacaaaaagtgtattaatgtatttgttcaggaaaagctacataccgaagggcttttgtatatgaattctgtggtggggagacccatttgtaatctatatggcagttccatctgggttttaagtttagatttcaccgtgtcttagtgcttcattctattggtttattggaacatgtaataaataggagtagtgatgtattaaaacacaagtattcattaatgttttatatcttcactaaaattctatagttatgaaactatcaatcaaggtgttatatttcagtcagaagtgaaaatttatgaagagtatttggaagtgtgtacagaaataaactagacttacaggtaggctagatcagaacgttaacatatgaacctgcagaaatctggtaagacttaaattcagtgtgaggaataactctagttctctcctatgagcatttcctaaaagccatctgatttggcattcttactggagctgcagacagaaatctacaaagacaaaagtaaacaaaattaagttattattccactgttaggaatggaaataaacttgtgaagtctgtttattttgaagtattggtgaactaggcttgctaattgataactgcagcagtttgtgtttactccagttcatcagcttaggtcatttgaaagatataagagcttaaggcaagaaagaaataacatggaattctatttgaaggacaacagaacattcttggaaaagcagctccagttggtttttcaactgtcaaacttgaatgtgtaagtccccacagagcatggacagtcggtgcagagttccaaggaaacaattattgcctgatgaccacttccattttgtatacactctttggttcgtataggccatattccaactggctttttagtaatagaaatccagtatataatgtatcaaatacaattgaggttctaacctagtgtgttaatttatctgaatttggatttttaaaaagtaataaaaagttaaatgta

SEQ ID NO: 6 - protein, 589 aa, Homo sapiens
  MISC_FEATURE (1)..(589): TM9SF3 protein
MRPLPGALGVAAAAALWLLLLLLPRTRADEHEHTYQDKEEVVLWMNTVGPYHNRQETYKYFSLPFCVGSKKSISHYHETLGEALQGVELEFSGLDIKFKDDVMPATYCEIDLDKEKRDAFVYAIKNHYWYQMYIDDLPIWGIVGEADENGEDYYLWTYKKLEIGFNGNRIVDVNLTSEGKVKLVPNTKIQMSYSVKWKKSDVKFEDRFDKYLDPSFFQHRIHWFSIFNSFMMVIFLVGLVSMILMRTLRKDYARYSKEEEMDDMDRDLGDEYGWKQVHGDVFRPSSHPLIFSSLIGSGCQIFAVSLIVIIVAMIEDLYTERGSMLSTAIFVYAATSPVNGYFGGSLYARQGGRRWIKQMFIGAFLIPAMVCGTAFFINFIAIYYHASRAIPFGTMVAVCCICFFVILPLNLVGTILGRNLSGQPNFPCRVNAVPRPIPEKKWFMEPAVIVCLGGILPFGSIFIEMYFIFTSFWAYKIYYVYGFMMLVLVILCIVTVCVTIVCTYFLLNAEDYRWQWTSFLSAASTAIYVYMYSFYYYFFKTKMYGLFQTSFYFGYMAVFSTALGIMCGAIGYMGTSAFVRKIYTNVKID

SEQ ID NO: 7 - DNA, 2391 bp, Homo sapiens; identical to SEQ ID NO: 3
  misc_feature (1)..(2391): TM9SF1encoding sequence

SEQ ID NO: 8 - protein, 606 aa, Homo sapiens
  MISC_FEATURE (1)..(606): TM1SF1 protein
MTVVGNPRSWSCQWLPILILLLGTGHGPGVEGVTHYKAGDPVILYVNKVGPYHNPQETYHYYQLPVCCPEKIRHKSLSLGEVLDGDRMAESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVERRSDRRRGDDGGFFPRTLEIHWLSIINSMVLVFLLVGFVAVILMRVLRNDLARYNLDEETTSAGSGDDFDQGDNGWKIIHTDVFRFPPYRGLLCAVLGVGAQFLALGTGIIVMALLGMFNVHRHGAINSAAILLYALTCCISGYVSSHFYRQIGGERWVWNIILTTSLFSVPFFLTWSVVNSVHWANGSTQALPATTILLLLTVWLLVGFPLTVIGGIFGKNNASPFDAPCRTKNIAREIPPQPWYKSTVIHMTVGGFLPFSAISVELYYIFATVWGREQYTLYGILFFVFAILLSVGACISIALTYFQLSGEDYRWWWRSVLSVGSTGLFIFLYSVFYYARRSNMSGAVQTVEFFGYSLLTGYVFFLMLGTISFFSSLKFIRYIYVNLKMD

SEQ ID NO: 9 - DNA, 20 bp, Artificial Sequence
  chemically synthesized
tgtgtgaaacaagcgccttc

SEQ ID NO: 10 - DNA, 19 bp, Artificial Sequence
  chemically synthesized
atgaggtggacgtagtagt

SEQ ID NO: 11 - DNA, 26 bp, Artificial Sequence
  chemically synthesized
gaattcatgtgtgaaacaagcgcctt

SEQ ID NO: 12 - DNA, 24 bp, Artificial Sequence
  chemically synthesized
gtcgacagaaaaccagtggatctg

SEQ ID NO: 13 - DNA, 18 bp, Artificial Sequence
  chemically synthesized
ccatggagaaggctgggg

SEQ ID NO: 14 - DNA, 20 bp, Artificial Sequence
  chemically synthesized
caaagttgtcatggatgacc

SEQ ID NO: 15 - DNA, 22 bp, Artificial Sequence
  chemically synthesized
gaattcatgtgtgaaacaagcg

SEQ ID NO: 16 - DNA, 24 bp, Artificial Sequence
  chemically synthesized
gtcgatgtctatcttcacagcata

SEQ ID NO: 17 - protein, 262 aa, Homo sapiens
  MISC_FEATURE (1)..(262): N-terminal domain corresponding to aa 18-279 TM9SF4
MCETSAFYVPGVAPINFHQNDPVEIKAVKLTSSRTQLPYEYYSLPFCQPSKITYKAENLGEVLRGDRIVNTPFQVLMNSEKKCEVLCSQSNKPVTLTVEQSRLVAERITEDYYVHLIADNLPVATRLELYSNRDSDDKKKEKDVQFEHGYRLGFTDVNKIYLHNHLSFILYYHREDMEEDQEHTYRVVRFEVIPQSIRLEDLKADEKSSCTLPEGTNSSPQEIDPTKENQLYFTYSVHWEESDIKWASRWDTYLTMSDVQIH

SEQ ID NO: 18 - protein, 15 aa, Homo sapiens
  MISC_FEATURE (1)..(15): Amino acids 221-235 of TM9SF4-protein
ADEKSSCTLPEGTNS

SEQ ID NO: 19 - protein, 126 aa, Homo sapiens
  MISC_FEATURE (1)..(126): Amino acids 90-215 of TM9SF1 -protein
ESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVER

SEQ ID NO: 20 - protein, 166 aa, Homo sapiens
  MISC_FEATURE (1)..(166): Amino acids 106-271 of TM9SF2 -protein
EPSPYKFTFNKKETCKLVCTKTYHTEKAEDKQKLEFLKKSMLLNYQHHWIVDNMPVTWCYDVEDGQRFCNPGFPIGCYITDKGHAKDACVISSDFHERDTFYIFNHVDIKIYYHVVETGSMGARLVAAKLEPKSFKHTHIDKPDCSGPPMDISNKASGEIKIAYTY

SEQ ID NO: 21 - protein, 194 aa, Homo sapiens
  MISC_FEATURE (1)..(194): amino acids 29-222 of TM9SF3-protein
DEHEHTYQDKEEVVLWMNTVGPYHNRQETYKYFSLPFCVGSKKSISHYHETLGEALQGVELEFSGLDIKFKDDVMPATYCEIDLDKEKRDAFVYAIKNHYWYQMYIDDLPIWGIVGEADENGEDYYLWTYKKLEIGFNGNRIVDVNLTSEGKVKLVPNTKIQMSYSVKWKKSDVKFEDRFDKYLDPSFFQHRIH

SEQ ID NO: 22 - DNA, 786 bp, Homo sapiens
  misc_feature (1)..(786): Nucleotides 287-1072 of  human tm9sf4 (NM_014742.3 GI164519075)
atgtgtgaaacaagcgccttctatgtgcctggggtcgcgcctatcaacttccaccagaacgatcccgtagaaatcaaggctgtgaagctcaccagctctcgaacccagctaccttatgaatactattcactgcccttctgccagcccagcaagataacctacaaggcagagaatctgggagaggtgctgagaggggaccggattgtcaacacccctttccaggttctcatgaacagcgagaagaagtgtgaagttctgtgcagccagtccaacaagccagtgaccctgacagtggagcagagccgactcgtggccgagcggatcacagaagactactacgtccacctcattgctgacaacctgcctgtggccacccggctggagctctactccaaccgagacagcgatgacaagaagaaggaaaaagatgtgcagtttgaacacggctaccggctcggcttcacagatgtcaacaagatctacctgcacaaccacctctcattcatcctttactatcatcgggaggacatggaagaggaccaggagcacacgtaccgtgtcgtccgcttcgaggtgattccccagagcatcaggctggaggacctcaaagcagatgagaagagttcgtgcactctgcctgagggtaccaactcctcgccccaagaaattgaccccaccaaggagaatcagctgtacttcacctactctgtccactgggaggaaagtgatatcaaatgggcctctcgctgggacacttacctgaccatgagtgacgtccagatccac

SEQ ID NO: 23 - DNA, 21 bp, Artificial Sequence
  chemically synthesized
atgtgtgaaacaagcgccttc

SEQ ID NO: 24 - DNA, 22 bp, Artificial Sequence
  chemically synthesized
gtggatctggacgtcactcatg

SEQ ID NO: 25 - DNA, 36 bp, Artificial Sequence
  chemically synthesized
caccaccacggcgtcatgtgtgaaacaagcgccttc

SEQ ID NO: 26 - DNA, 42 bp, Artificial Sequence
  chemically synthesized
cgagcgaaggcgtcagattagtggatctggacgtcactcatg

SEQ ID NO: 27 - protein, 16 aa, Artificial
  Chemically synthesized
MGSDKIHHHHHHHHHH

SEQ ID NO: 28 - DNA, 378 bp, Homo sapiens
  misc_feature (1)..(378): Nucleotides 616-993 of human TM9SF1 (NM_006405.5; GI62460631 variant 1; NM_00204842.1; GI62460634 variant 2)
gagtctttgtatgagatccgctttcgggaaaacgtggagaagagaattctgtgccacatgcagctcagttctgcacaggtggagcagctgcgccaggccattgaagaactgtactactttgaatttgtggtagatgacttgccaatccggggctttgtgggctacatggaggagagtggtttcctgccacacagccacaagataggactctggacccatttggacttccacctagaattccatggagaccgaattatatttgccaatgtttcagtgcgggacgtcaagccccacagcttggatgggttacgacctgacgagttcctaggccttacccacacttatagcgtgcgctggtctgagacttcagtggagcgt

SEQ ID NO: 29 - DNA, 21 bp, Artificial Sequence
  chemically synthesized
gagtctttgtatgagatccgc

SEQ ID NO: 30 - DNA, 18 bp, Artificial Sequence
  chemically synthesized
acgctccactgaagtctc

SEQ ID NO: 31 - DNA, 498 bp, Homo sapiens
  misc_feature (1)..(498): Nucleotides 449-946 of human tm9SF3 (NM_004800.1 GI4758873)
gaaccttcaccatataagtttacgtttaataagaaggagacctgtaagcttgtttgtacaaaaacataccatacagagaaagctgaagacaaacaaaagttagaattcttgaaaaaaagcatgttattgaattatcaacatcactggattgtggataatatgcctgtaacgtggtgttacgatgttgaagatggtcagaggttctgtaatcctggatttcctattggctgttacattacagataaaggccatgcaaaagatgcctgtgttattagttcagatttccatgaaagagatacattttacatcttcaaccatgttgacatcaaaatatactatcatgttgttgaaactgggtccatgggagcaagattagtggctgctaaacttgaaccgaaaagcttcaaacatacccatatagataaaccagactgctcagggccccccatggacataagtaacaaggcttctggggagataaaaattgcctatacttac

SEQ ID NO: 32 - DNA, 29 bp, Artificial Sequence
  chemically synthesized
gaaccttcaccatataagtttacgtttaa

SEQ ID NO: 33 - DNA, 27 bp, Artificial Sequence
  chemically synthesized
gtaagtataggcaatttttatctcccc

SEQ ID NO: 34 - DNA, 582 bp, Homo sapiens
  misc_feature (1)..(582): Nucleotides 301-883 of human tm9sf3 (NM_020123.3 GI190104385)
gacgagcacgaacacacgtatcaagataaagaggaagttgtcttatggatgaatactgttgggccctaccataatcgtcaagaaacatataagtacttttcacttccattctgtgtggggtcaaaaaaaagtatcagtcattaccatgaaactctgggagaagcacttcaaggggttgaattggaatttagtggtctggatattaaatttaaagatgatgtgatgccagccacttactgtgaaattgatttagataaagaaaagagagatgcatttgtatatgccataaaaaatcattactggtaccagatgtacatagatgatttaccaatatggggtattgttggtgaggctgatgaaaatggagaagattactatctttggacctataaaaaacttgaaataggttttaatggaaatcgaattgttgatgttaatctaactagtgaaggaaaggtgaaactggttccaaatactaaaatccagatgtcatattcagtaaaatggaaaaagtcagatgtgaaatttgaagatcgatttgacaaatatcttgatccgtccttttttcaacatcggattcat

SEQ ID NO: 35 - DNA, 22 bp, Artificial Sequence
  chemcially synthesized
gacgagcacgaacacacgtatc

SEQ ID NO: 36 - DNA, 22 bp, Artificial Sequence
  chemically synthesized
atgaatccgatgttgaaaaaag

SEQ ID NO: 37 - RNA, 25 nt, artificial sequence
  chemically synthetized
gagugacguccagauccacugguuu

SEQ ID NO: 38 - RNA, 25 nt, artificial sequence
  chemically synthetized
aaaccaguggaucuggacgucacuc

SEQ ID NO: 39 - protein, 606 aa, Homo sapiens; identical to SEQ ID NO: 8
  MISC_FEATURE (1)..(606): TM9SF 1 isoform CRA_c (GI119586480)

SEQ ID NO: 40 - protein, 462 aa, Homo sapiens
  MISC_FEATURE (1)..(462): TM9SF1 isoform CRA_b (GI119586478)
MEESGFLPHSHKIGLWTHLDFHLEFHGDRIIFANVSVRDVKPHSLDGLRPDEFLGLTHTYSVRWSETSVERRSDRRRGDDGGFFPRTLEIHWLSIINSMVLVFLLVGFVAVILMRVLRNDLARYNLDEETTSAGSGDDFDQGDNGWKIIHTDVFRFPPYRGLLCAVLGVGAQFLALGTGIIVMALLGMFNVHRHGAINSAAILLYALTCCISGYVSSHFYRQIGGERWVWNIILTTSLFSVPFFLTWSVVNSVHWANGSTQALPATTILLLLTVWLLVGFPLTVIGGIFGKNNASPFDAPCRTKNIAREIPPQPWYKSTVIHMTVGGFLPFSAISVELYYIFATVWGREQYTLYGILFFVFAILLSVGACISIALTYFQLSGEDYRWWWRSVLSVGSTGLFIFLYSVFYYARRSNMSGAVQTVEFFGYSLLTGYVFFLMLGTISFFSSLKFIRYIYVNLKMD

SEQ ID NO: 41 - protein, 489 aa, Homo sapiens
  MISC_FEATURE (1)..(489): TM9SF1 isoform CRA_c (GI11958647)
MTVVGNPRSWSCQWLPILILLLGTGHGPGVEGVTHYKAGDPVILYVNKVGPYHNPQETYHYYQLPVCCPEKIRHKSLSLGEVLDGDRMAESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVERRSDRRRGDDGGFFPRTLEIHWLSIINSMVLVFLLVGFVAVILMRVLRNDLARYNLDEETTSAGSGDDFDQGDNGWKIIHTDVFRFPPYRGLLCAVLGVGAQFLALGTGIIVMALLGMFNVHRHGAINSAAILLYALTCCISGYVSSHFYRQIGGERWVWNIILTTSLFSVPFFLTWSVVNSVHWANGSTQALPATTILLLLTVWLLVGFPLTVIGGIFGKNNASPFDAPCRTKNIAREIPPQPWYKSTVIHMTVGGFLPFRYPPFIPWLLLSGS

SEQ ID NO: 42 - protein, 519 aa, Homo sapiens
  MISC_FEATURE (1)..(519): TM9SF1 isoform CRA_d (GI119586481)
MAESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVERRSDRRRGDDGGFFPRTLEIHWLSIINSMVLVFLLVGFVAVILMRVLRNDLARYNLDEETTSAGSGDDFDQGDNGWKIIHTDVFRFPPYRGLLCAVLGVGAQFLALGTGIIVMALLGMFNVHRHGAINSAAILLYALTCCISGYVSSHFYRQIGGERWVWNIILTTSLFSVPFFLTWSVVNSVHWANGSTQALPATTILLLLTVWLLVGFPLTVIGGIFGKNNASPFDAPCRTKNIAREIPPQPWYKSTVIHMTVGGFLPFSAISVELYYIFATVWGREQYTLYGILFFVFAILLSVGACISIALTYFQLSGEDYRWWWRSVLSVGSTGLFIFLYSVFYYARRSNMSGAVQTVEFFGYSLLTGYVFFLMLGTISFFSSLKFIRYIYVNLKMD

SEQ ID NO: 43 - protein, 489 aa, Homo sapiens; identical to SEQ ID NO: 41
  MISC_FEATURE (1)..(489): TM9SF1 isoform b (GI 62460635)

(19) United States
(12) Patent Application Publication
(10) Pub. No.: US 2012/0122118 A1
(43) Pub. Date: May 17, 2012

(54) Monclonal antibodies, hybridomas and methods for use

(75) Inventors: Francesco Lozupone, Rome (IT); Stefano Fais, Rome (IT); Antonio Chiesi, Vigasio (IT); Natasa Zarovni, Milan (IT); Angela Simona Pontillo, Milan (IT); Paolo Sarmientos, Milan (IT)

(73) Assignees: PRIMM SRL, Milan (IT); HANSABIOMED OU, Tallin (EE)

(21) Appl. No.: 13/290,186

(22) Filed: Nov. 7, 2011

Related U.S. Application Data
(63) Continuation-in-part of application No. 12/321,821, filed on Jan. 26, 2009, now Pat. No. 8,097,407.
(60) Provisional application No. 61/062,453, filed on Jan. 25, 2008.

(30) Foreign Application Priority Data
Jan. 26, 2009 (AU) 2009207926
Jan. 26, 2009 (BR) BR P 10906081-0
Jan. 26, 2009 (CA) CA2713193
Jan. 26, 2009 (EE) PCT/EE2009/000001
Jan. 26, 2009 (EP) EP09703627
Jan. 26, 2009 (JP) JP2010543378
Jan. 26, 2009 (MX) MX/A/2010/008164
Jan. 26, 2009 (RU) 2010135525

(51) Int. Cl. G01N 33/566; C07K 16/30; C07K 17/00; C12N 5/18

(52) U.S. Cl. 435/7.5; 435/331; 530/387.9; 530/391.3; 530/391.7; 435/7.92

(57) ABSTRACT

The present invention provides monoclonal antibodies to TM9SF-proteins and hybridoma cell lines that produce the monoclonal antibodies to TM9SF4. This invention also provides a method for determining the level of TM9SF4-protein in biological fluid samples, tissue samples and in microvesicles such as exosomes, comprising kit for determining the level of TM9SF4 protein in human exosomes and other microvesicles, in tissue samples, and in biological fluids.

### PRIORITY

This application is a continuation in part application of U.S. application Ser. No. 12/321,821 filed on Jan. 26, 2009 which claims priority of the U.S. provisional application No. 61/062,528 filed on Jan. 25, 2008, both of which are incorporated by reference herein in their entirety.

### SEQUENCE DATA

This application contains sequence data provided on a computer readable diskette and as a paper version. The paper version of the sequence data is identical to the data provided on the diskette.

### COLOR DRAWINGS

This patent application contains at least one drawing executed in color. Copies of this patent or patent application publication with color drawing(s) will be provided by the Office upon request and payment of the necessary fee.

### FIELD OF THE INVENTION

The present invention relates generally to the field of cancer research, diagnosis and prognosis. More specifically the invention relates to production and use of monoclonal antibodies. More specifically this invention is related to monoclonal antibodies against TM9SF-proteins and hybridomas producing the antibodies.

### BACKGROUND OF THE INVENTION

Cancer is a major public health problem in the United States and many other parts of the world. Currently, one in four deaths in the United States is due to cancer. Early diagnosis improves significantly patient's survival and the finding of new and/or more specific cancer markers is one of the major endpoints in the fight against cancer. The identification of new tumor markers may be extremely helpful not only in tumor diagnosis, but also as potential new targets for anti-tumor strategies, possibly more effective and less toxic than current standard therapies.

Pathological research provides means for establishing the diagnosis of most solid tumors. Although many cases can be classified reliably with current pathological criteria, there is still a significant subset of cases in which no consensus can be reached even among expert pathologists. Diagnostic ambiguity has significant adverse consequences for the patient. Misclassifying a tumor as benign may be fatal, and diagnosing a benign lesion as malignant may lead to unnecessary treatments. Currently there is no method to definitely resolve these ambiguities. Therefore, there is a clear need for a diagnostic test that could reduce these uncertainties.

Phagocytosis is the process by which cells internalize large particles (typically 0.1 mm diameter), such as bacteria or cell debris. The early stage of phagocytosis can be tentatively divided into distinctive steps: cell membrane binding around the particle, phagosome formation, and internalization of the phagosome. In the process of phagosome formation and internalization, actin cytoskeleton has been proposed to drive these steps to allow engulfment.

Phagocytic cells have been identified in malignant tumors over a century ago. More recently, cells with phagocytic behavior (also defined as cannibalistic behavior) have been detected in tumors of differing histologies, such as oat cell carcinoma of the lung, breast cancer, bladder cancer, medulloblastoma, gastric adenocarcinomas, melanoma and squamous cell carcinoma of the skin.

We have recently observed that phagocytosis is a character of metastatic melanoma cells able to phagocytose apoptotic cells, plastic beads stained yeasts, and live lymphocytes displaying efficient phagocytic machinery responsible for a macrophage-like activity, while melanoma cells derived from primary lesions did not display any cannibalistic or phagocytic activity. Moreover, cannibal cells can be detected in 100% metastatic melanoma lesions (Lugini et al., 2004; Lugini et al., 2006).

One of the main features of cannibal cells is an increased acidity of lysosomal-like vesicles and an over expression of cathepsin B, a proteolytic enzyme reported to be involved in tumor invasion and metastasis (Sloane et al., 1981). Different from professional phagocyte-like macrophages, cannibal tumor cells do not utilize structures like ruffles or any pseudopodial movement. Instead, live or dead material that touches the tumor cell's external membrane is immediately endocytosed and digested through a sort of quicksand mechanism that seems not to involve any specific receptor.

These findings have led us to speculate that cannibal cells feed off other cells, perhaps with no particular need of a blood-derived nutrient supply, but also that cannibalism of lymphocytes by tumor cells may represent a rudimentary mechanism of tumor immune escape. Moreover, these findings led us to a novel, revolutionary interpretation that cancer cells, in their habit to use other cells for feeding, may behave as unicellular eukaryotes whose unique purpose is to survive in a continuous fighting against other cells and the unfavourable environment. This theory further led us to speculate that amoebas and metastatic cells might share the same framework with the same regulatory elements allowing their surviving in adverse micro-environmental conditions. However, so far no genes have ever been specifically associated with the cannibal behaviour of cancer cells.

The cellular slime mold Dictyostelium discoideum has been previously used as a model organism to study phagocytosis. Mechanisms involved in phagocytosis by Dictyostelium cells are very similar to those used by mammalian phagocytes, and involve the actin cytoskeleton and RacF1, a member of the Rho family of GTP-binding proteins. However, no phagocytosis associated specific proteins have ever been identified in mammals.

It has been recently found that the protein encoded by phg1A-gene was implicated in cell adhesion and phagocytosis in the amoeba Dictyostelium discoideum. This protein belongs to TransMembrane 9 Super Family (TM9SF) and genes encoding TM9-proteins can be unambiguously identified in eukaryotic genomes. The family includes many members in organisms ranging from yeast to plants and human. To mention some example, there are three members of this family in Saccharomyces cerevisiae, Dictyostelium amoebae, and Drosophila flies and four in humans and mice. All of them exhibit a similar overall structure, with a rather variable potential luminal domain followed by a more conserved membrane domain and nine or ten putative transmembrane domains.

TransMembrane 9 SuperFamily (TM9SF1, TM9SF2, TM9SF3, TM9SF4/TUCAP1) is a very closely related family of proteins with a high degree of homology. This family of proteins is characterized by the presence of a large variable extracellular or lumenal N-terminal domain followed by nine putative transmembrane domains in its conserved C-terminal. These proteins are almost completely uncharacterized. The only data available describes TM9SF1 as a protein involved in the autophagic processes, and seems to be differentially expressed in urinary bladder cancer [1-2]. TM9SF3 is upregulated in Paraclitaxel resistant breast cancer cells [3]. Finally TM9SF4 involved in myeoloid malignancy [4]. WO2100/022387 describes TM9SF-proteins as markers for aging related disorders.

TM9SF4 has been characterized for the first time in U.S. Serial Number 2009/019122, which is incorporated herein by reference, and in a subsequent publication by Lozupone et al, 2009 [5] also incorporated herein by reference, where this protein has been described as a new tumor associated protein, highly expressed in metastatic melanoma cells, while undetectable in skin cells and peripheral blood lymphocytes derived from healthy donors. In the same publication the authors show that TM9SF4 is clearly involved in the development of the cannibal behavior of metastatic melanoma cells. The protein was named as TUCAP-1, which is used in this disclosure as a synonym for TM9SF4-protein. Tumor cell cannibalism is a phenomenon, characterized by the ability of tumor cannibal cells to phagocyte apoptotic cells, plastic beads, stained yeasts as well as live lymphocytes. This phenomenon has been observed in tumors of different histology, and always related to a poor prognosis [6-11].

TM9SF4 subcellular localization analysis suggests that this protein is mainly recovered in intracellular vesicles such as early endosomes, since it co-localizes with early endosomal markers such as Rab5 and EEA1. Moreover the predicted structure of TM9SF4 makes conceivable to hypothesize a role for this molecule as an ion channel or an ion channel regulatory protein involved in pH regulation of intracellular vesicles. Literature about ion channels and intracellular pH alteration widely suggest that ion channels or proton pumps and, more in general, proteins involved in the intracellular pH regulation of cancer cells have a role in the malignant behavior of many tumors [12-17]. TM9SF4 localization, its role in endosomal pH regulation, and its structure suggesting that TM9SF4 could be an ion channel or an ion channel interacting protein, allowed us to suppose that by TM9SF4 could play a role in drug resistance through the deregulation of intracellular cellular pH.

To address the currently existing problems in the art, the present disclosure enables generation of antibodies and antibody preparations by using antigen sequences of TM9SF-proteins. The disclosure provides a variety of uses for the antibodies and the antibody-preparations.

### SUMMARY OF THE INVENTION

Based on our theory that cancer cells use other cells for feeding and behaving as unicellular eukaryotes and possibly share the same framework with the same regulatory elements as amoebas, we compared phg1A-gene with human genome. Three homologues of phg1 have been fully sequenced in human (TM9SF4, U81006 and U9483 1), and we found the closest homologue of phg1 of Dictyostelium dicoideum in human to be tm9sf4 (other aliases: KIAA0255, dJ836N17.2) located in chromosome 20q11.21. Even if this gene is fully sequenced, our disclosure in U.S. Serial Number 2009/019122 and in the corresponding provisional application 61/062,528, which are fully incorporated herein by reference, described for the first time the function and expression product of this protein.

An object of this disclosure is to provide antibodies, primers, oligopeptides and polypeptides useful for TM9SF4 (TUCAP) detection, analysis and potential therapeutic applications.

Another object of this disclosure is to provide hybridomas to produce the monoclonal antibodies against TM9SF4.

Yet another object is to provide antibodies that bind to TM9SF4-protein and polypeptide fragments thereof, including polyclonal and monoclonal antibodies, murine and other mammalian antibodies, chimeric antibodies, humanized and fully human antibodies, and antibodies labelled with a detectable marker, and antibodies conjugated to radionucleotides, toxins or other therapeutic compositions. The invention further provides methods for detecting the presence of TM9SF4-polypeptides and proteins in various biological samples, as well as methods for identifying cells that express TM9SF4.

Another object of this invention is to provide mouse anti-TM9SF4 monoclonal antibodies, hybridomas for producing the antibodies, an improved method for determining i) the circulating TM9SF4 protein or free TM9SF4 protein domains, ii) TM9SF4 protein or fragments expressed in exosomes or other microvesicles deriving from body fluids and cultured cells supernatants and the kits for performing said determinations.

Yet another object of this invention is to provide anti-TM9SF4 monoclonal antibodies, fragments thereof or antibody preparations for use in diagnostics and prognosis of tumors as well as in cancer treatments.

Still another object of this invention is to provide methods and kits for the determination of the level of TM9SF4 protein or TM9SF4 protein fragments, in tissue samples, biological fluids, and exosomes.

A further object of this invention is to provide antibodies against other members of TM9-superfamily, including TM9SF1, TM9SF2 and TM9SF3.

An even further object of this invention is to provide methods and kits for the determination of the level of TM9-superfamily proteins, including TM9SF1, TM9SF2 and TM9SF3, or protein fragments, in tissue samples, biological fluids, and on exosomes.

Yet another object of this invention is to provide a method for early diagnosis or prognosis of cancer in human subjects.

An object of this invention is to provide a hybridoma cell line selected from the group consisting of hybridoma cell lines 1A4-A3, 1A4-A8, 1A4-F2, 1A4-G1, 5C1-B4, 5C1-C5, 5C1-D4, and 5C1-G6.

Another object of this invention is to provide an isolated monoclonal antibody or a fragment thereof, wherein the antibody is produced by the hybridoma cell line selected from the group consisting of hybridoma cell lines 1A4-A3, 1A4-A8, 1A4-F2, 1A4-G1, 5C1-B4, 5C1-C5, 5C1-D4, and 5C1-G6.

### SHORT DESCRIPTION OF THE DRAWINGS

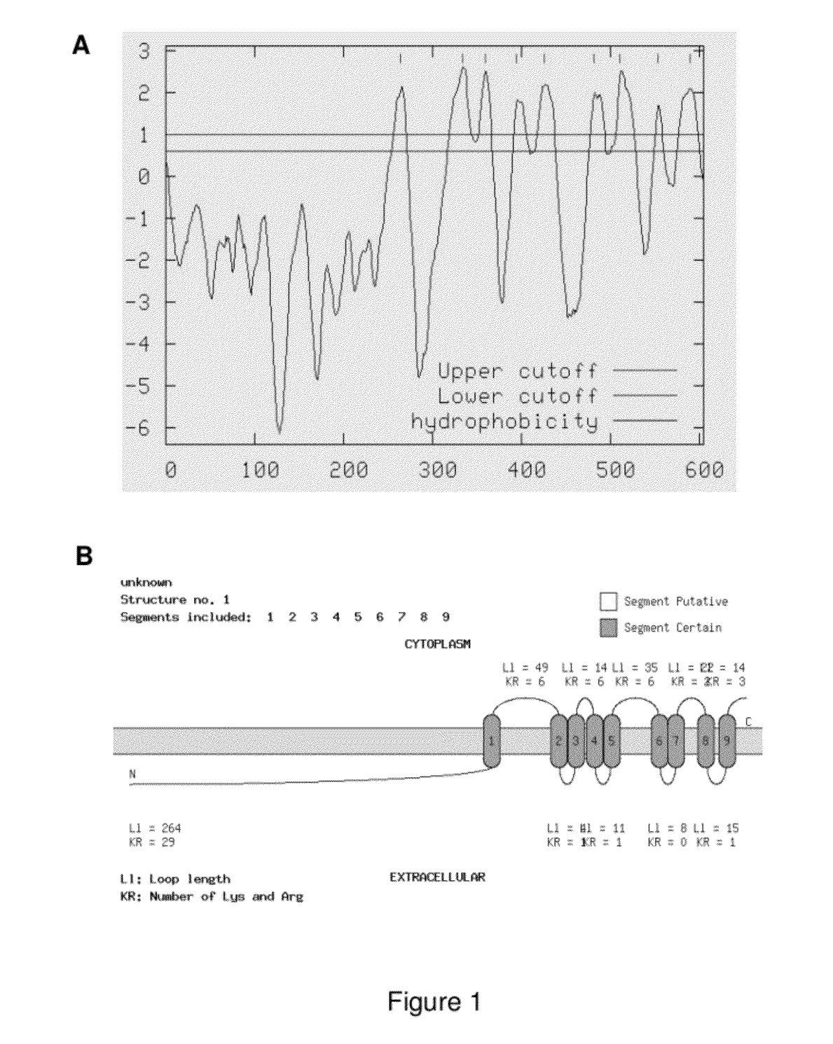

FIG. 1 Molecular structure of TM9SF4/TUCAP-1 protein.

(A) Hydropathy profile of TM9SF4/TUCAP-1 protein sequence. Hydrophobic regions are indicated above the line by positive values. Amino acid numbering is indicated on the abscissa. Hydrophilic stretch in the N-terminal region is followed by nine hydrophobic regions. Analysis was performed according to Claros and von Heijneb using TopPred prediction Program.
(B) Graphic representation of TM9SF4/TUCAP-1 secondary structure according to TopPred predictor server.

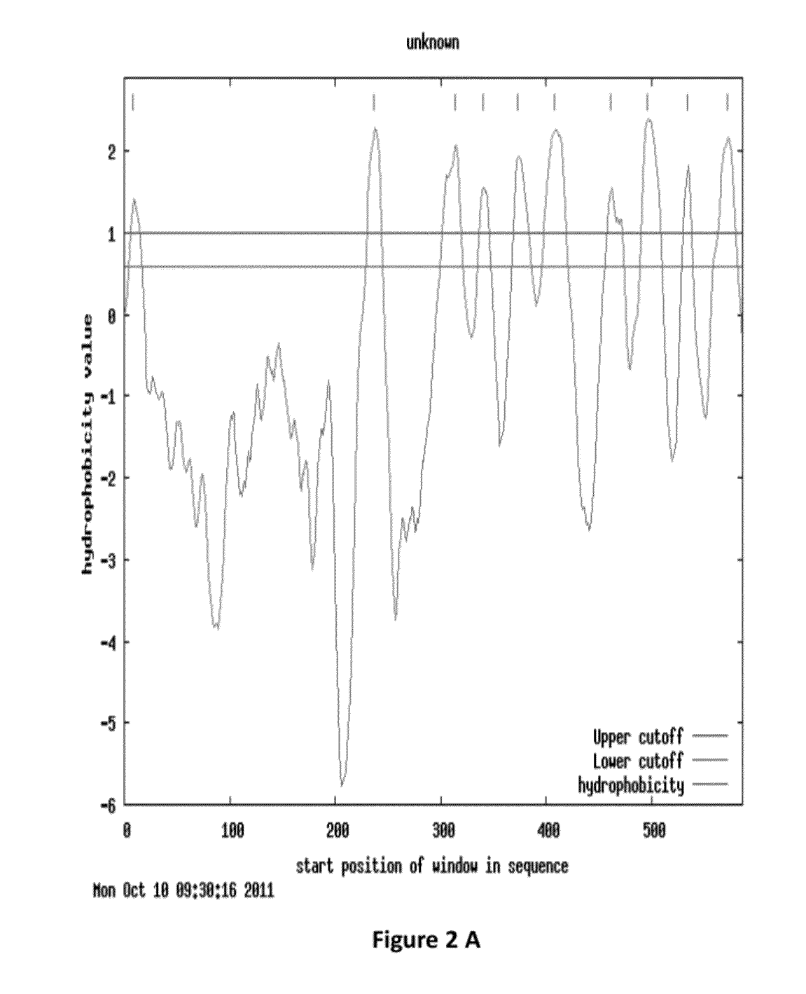

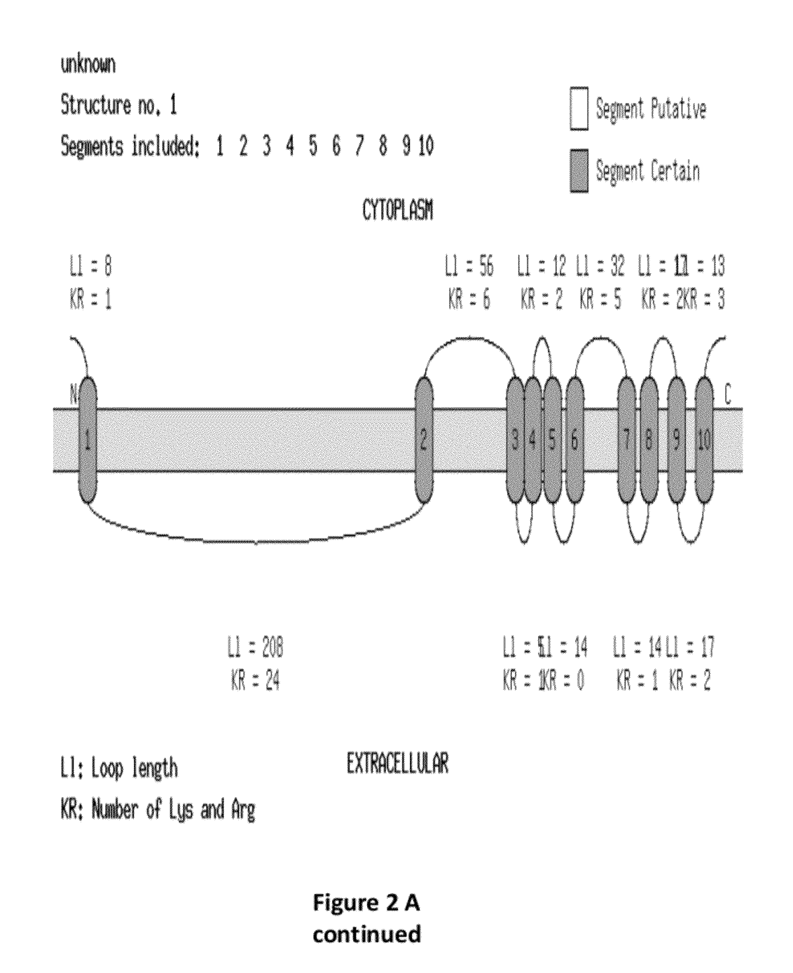

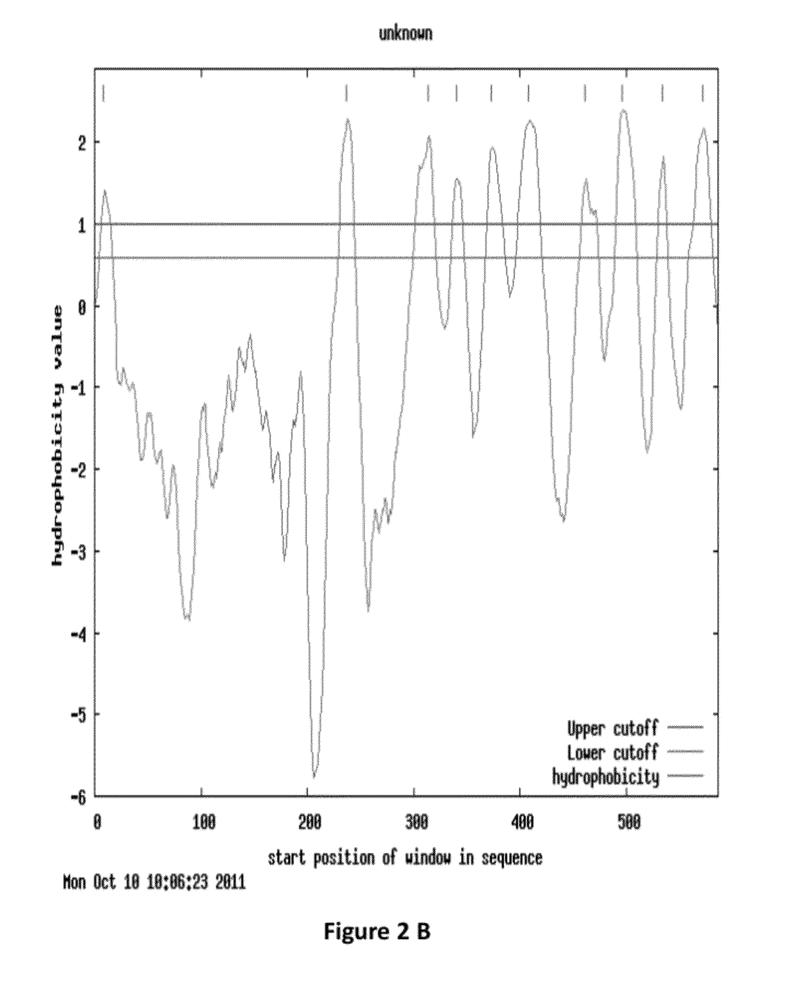

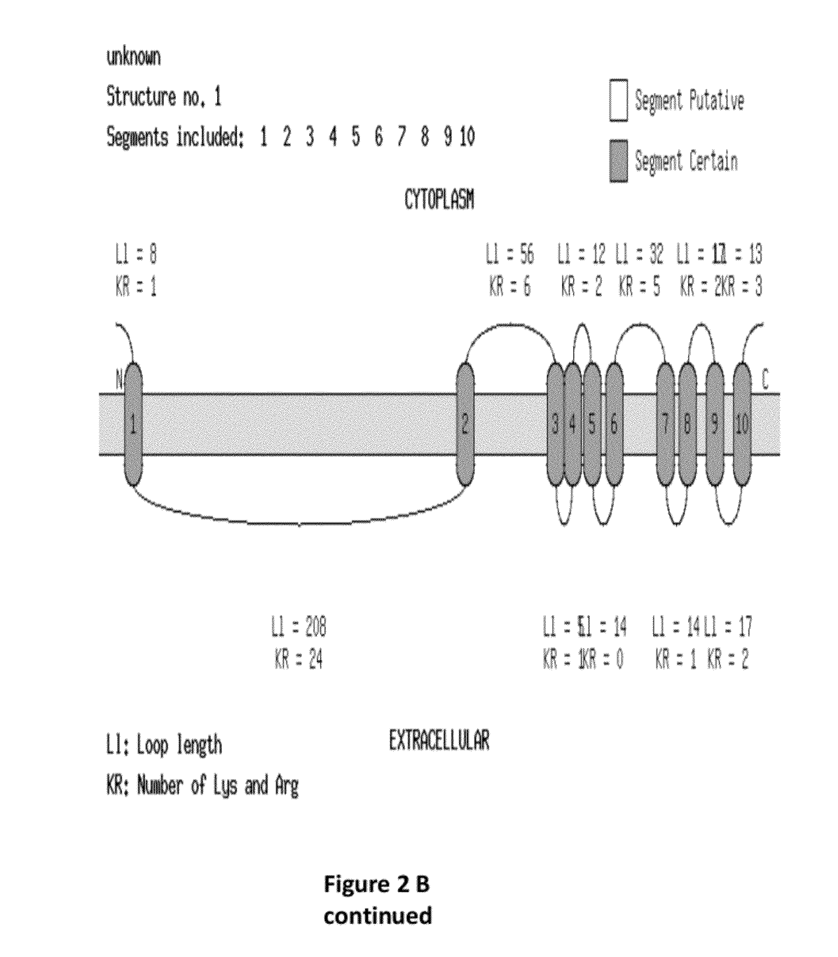

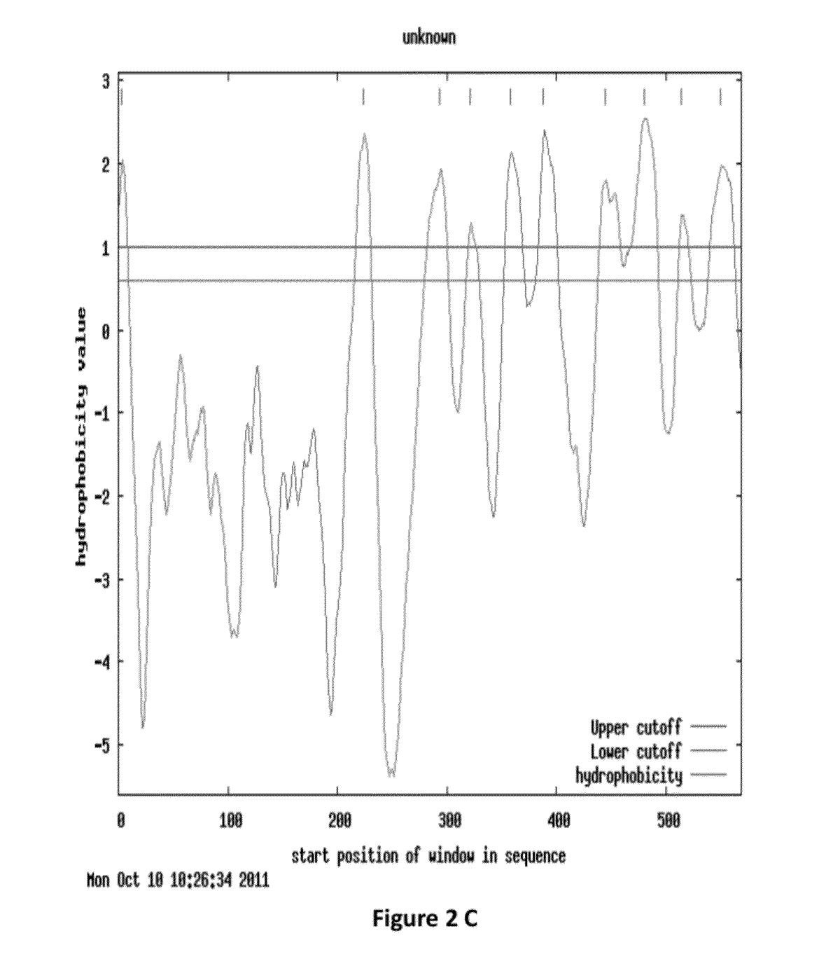

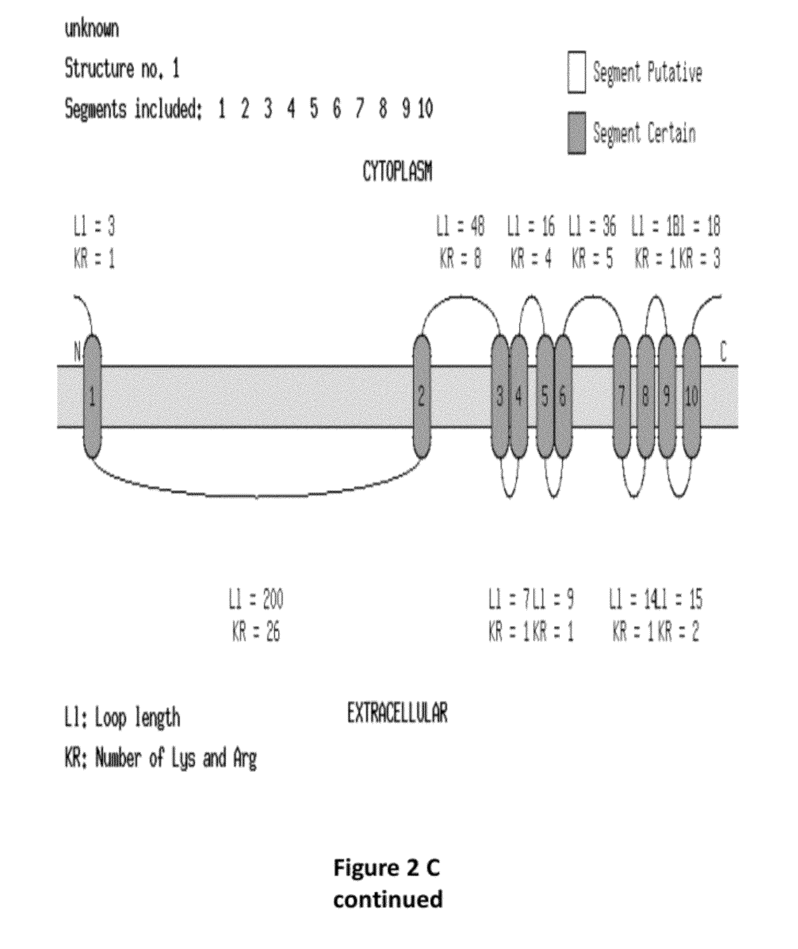

FIG. 2 Molecular structure of TM9SF1-3 proteins. Hydrophobic regions are indicated above the line by positive values. Amino acid numbering is indicated on the abscissa. Hydrophilic stretch in the N-terminal region is followed by nine hydrophobic regions. Analysis was performed according to Claros and von Heijneb using TopPred prediction server.
A. Hydropathy profile of TM9SF1 protein sequence (left) and Graphic representation of TM9SF1 secondary structure (right) according to TopPred predictor server.
B. Hydropathy profile of TM9SF2 protein sequence (left) and Graphic representation of TM9SF2 secondary structure (right) according to TopPred predictor server
C. Hydropathy profile of TM9SF3 protein sequence (left) and Graphic representation of TM9SF3 secondary structure (right) according to TopPred predictor server

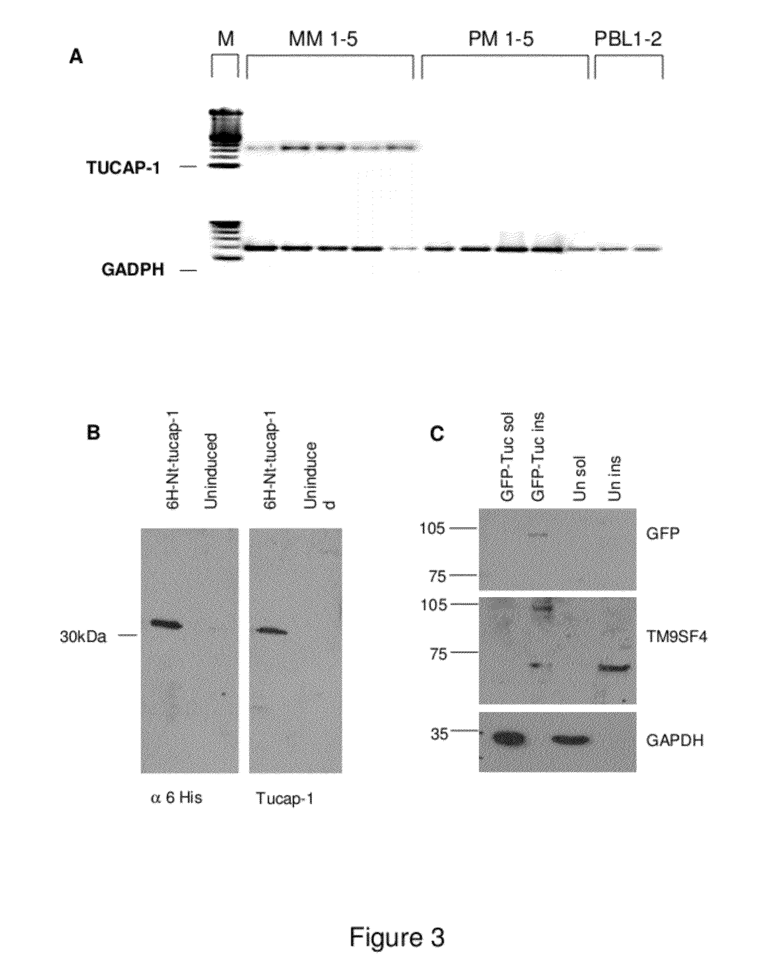

FIG. 3. Detection of TM9SF4/TUCAP-1 transcripts and characterization of TUCAP-1-antibodies:

(A) RT-PCR analysis of TUCAP-1 (upper panel) and GAPDH (lower panel) on five metastatic (MM1-5) and five primary melanoma (PM1-5) cell lines recently established in vitro from metastatic lesion, and peripheral blood cells from two different donors (PBL1-2); M size marker.
(B) Western blots of 6-histidine or TUCAP-1 in the six-histidine tagged TUCAP-1 peptide used to immunize mice and in uninduced bacterial lysates. Equal amount of purified protein and bacterial lysate was loaded on reducing gels and blotted with the 6-His-antibody or TUCAP-1 mice antisera. Proteins were visualized using HRP-conjugated secondary antibodies and revealed with ECL system (Pierce).
C. Western blotting for GFP-TUCAP-1 and GADPH on Triton soluble (lanel) and Triton insoluble (lane 2) fraction of GFP-TUCAP-1 (GFP-Tuc) transfected MM1 cells, and Triton soluble and insoluble fractions of untransfected MM1 cells (lanes 3-4). M is a size marker. Proteins were visualized using HRP conjugated secondary antibodies and revealed with ECI (Pierce). As molecular weicht markers Rainbow™ (Amersham UK) prestained standards were used.

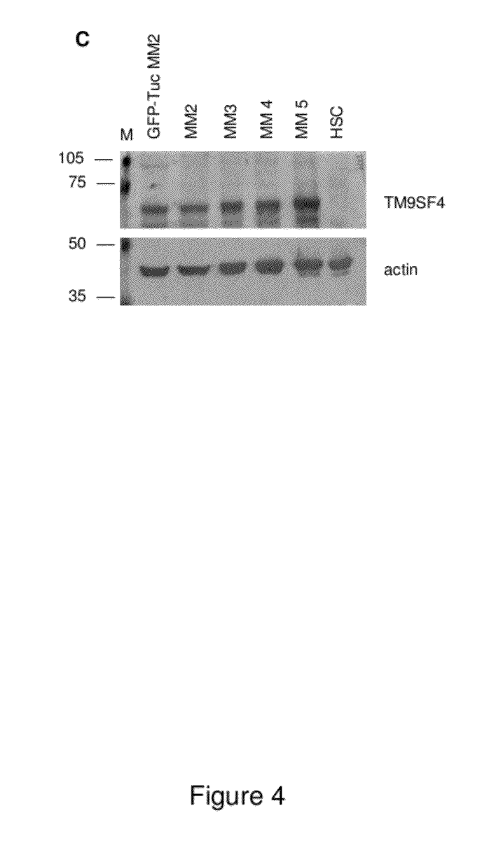

FIG. 4. Western blotting analysis of TM9SF4/TUCAP-1.

Western blotting for TUCAP-1 detection on GFP-Tuc Transfected MM2 cells,f our metastatic melanoma cell lines (MM2-5), and CCD-1064SK human skin fibroblasts (HSC). Loading amount was controlled by immunodetection of actin. Proteins were visualized using HRP conjugated secondary antibodies and DAB system (DAKO, Denmark) as cromogen. Rainbow™ (Amersham™ UK) prestained standards were used as molecular weight markers.

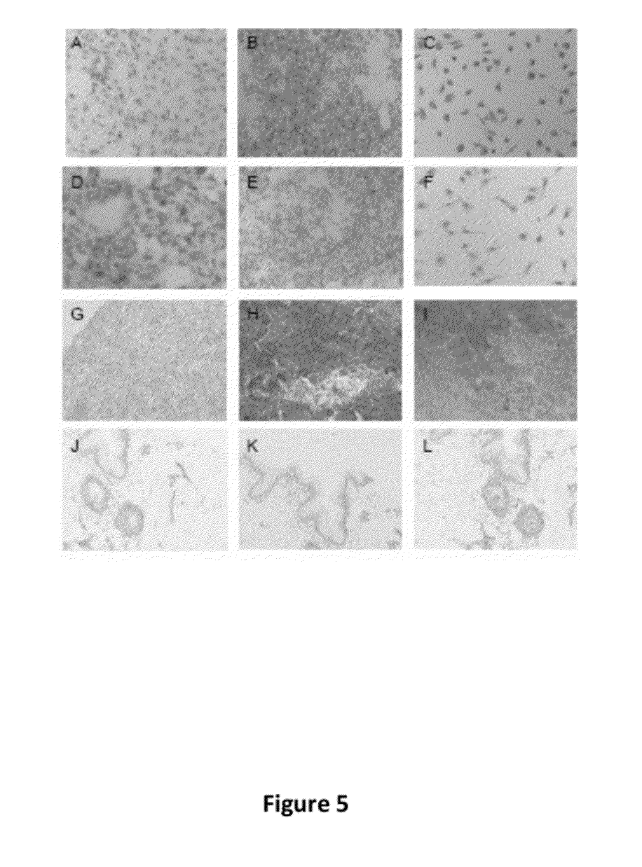

FIG. 5. Immuno-cytochemical and immuno-histochemical analysis of TM9SF4/TUCAP-1.

Mice pre-immune serum immunocytochemical analysis of (A) MM2 cells; (B) peripheral blood lymphocytes; (C) in vitro differentiated macrophages.
TUCAP-1 immunocytochemical analysis of: (D) M2 cells; (E) peripheral blood cells; (F) Macrophages.
Immunohistochemical analysis of malignant melanoma tissues stained with: (G) preimmune mouse serum; (H) TUCAP-1-immune serum; and (1) anti-GP100.
Immunohistochemical analysis of healthy skin stained with: (J) mouse preimmune serum, (K) TUCAP-1 immune serum, and (L) anti-ezrin antibody. Magnification 10×.

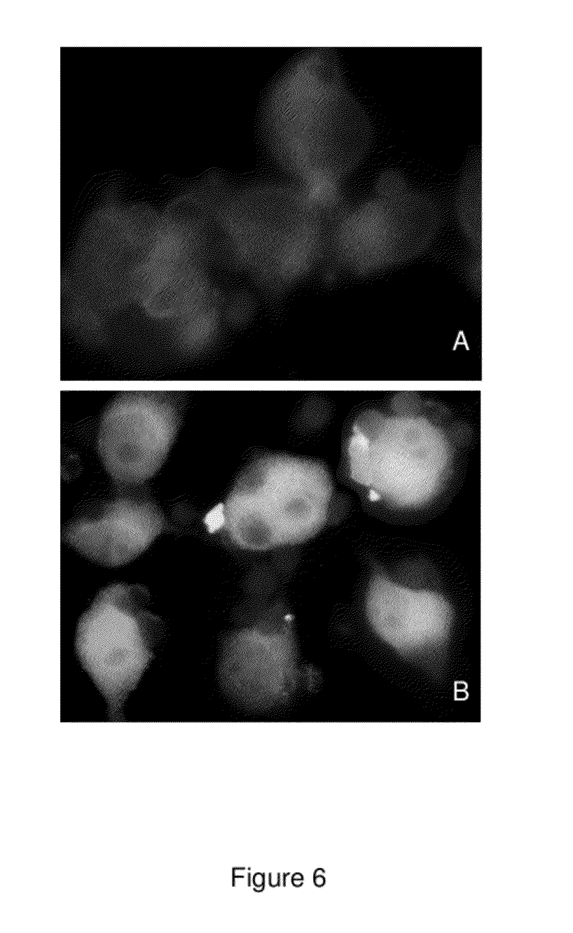

FIG. 6. TMSF4/TUCAP-1-detection in cannibal cells

(A) Detection and localization of TUCAP-1 in metastatic melanoma MM1 cells co-cultured with living lymphocytes. IVM analysis of TUCAP-1 (green). Picture highlights that TUCAP-1 is detectable exclusively on melanoma cells.
(B) Double fluorescence analysis of TUCAP-1 (green) and EEA-1 (red) in metastatic melanoma MM1 cells co-cultured with living lymphocytes. Yellow/orange areas indicate co-localization. Nuclei are stained with Hoechst 33258.

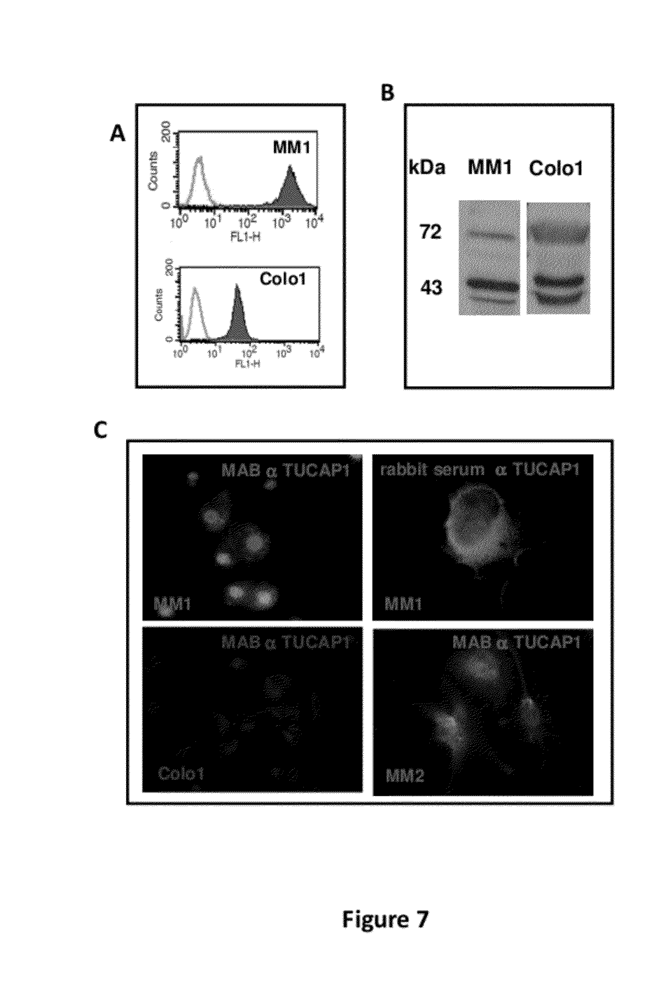

FIG. 7. Expression of TM9SF4 on MM1 and Colo1 tumor cells
A. FACS analysis of TM9SF4 expression on MM1 and Colo1 cells, Green line: negative control, Purple: TM9SF4 protein.
B. Western Blot analysis of MM1 and Colo1 whole cell lysates immunoblotted with rabbit polyclonal anti TM9SF4 serum.
C. Immunofluorescence analysis of MM1 cells stained with either monoclonal or polyclonal TM9SF4 antibodies and MM2 and Colo1 cells stained with monoclonal anti TM9SF4 antibody. As negative controls control isotype antibodies were used.

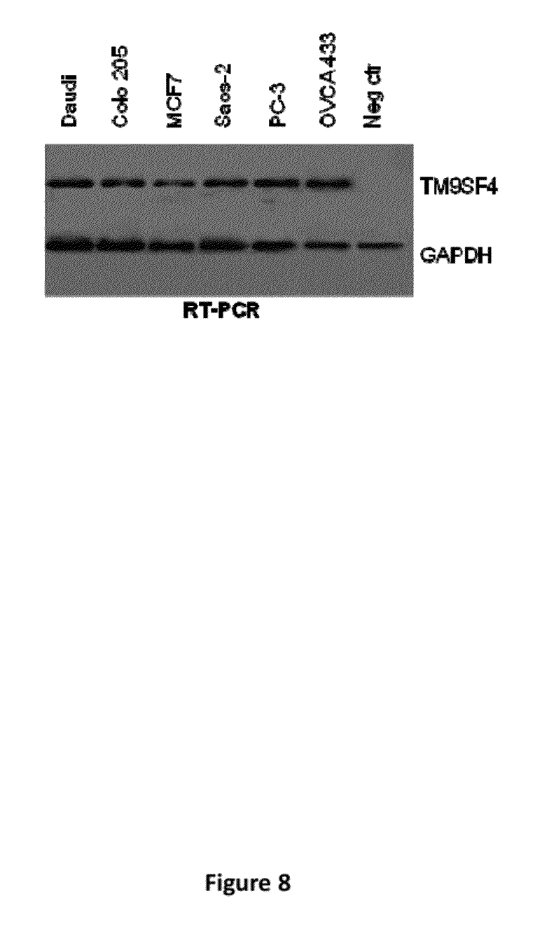

FIG. 8 RT-PCR analysis of TM9SF4 on different tumor cell lines.
The expression of TUCAP1/TM9SF4 was evaluated by RT-PCR on different cell lines. The data is representative of B lymphoma (Daudi); Colon Carcinoma (Colo 205); breast carcinoma (MCF7); Osteosarcoma (Saos-2); prostate cancer (PC-3); and ovary carcinoma (OVCA 433). Metastatic melanoma MM1 cells as positive control were used. As negative control template without reverse transcriptase was used. As housekeeping gene GAPDH was used.

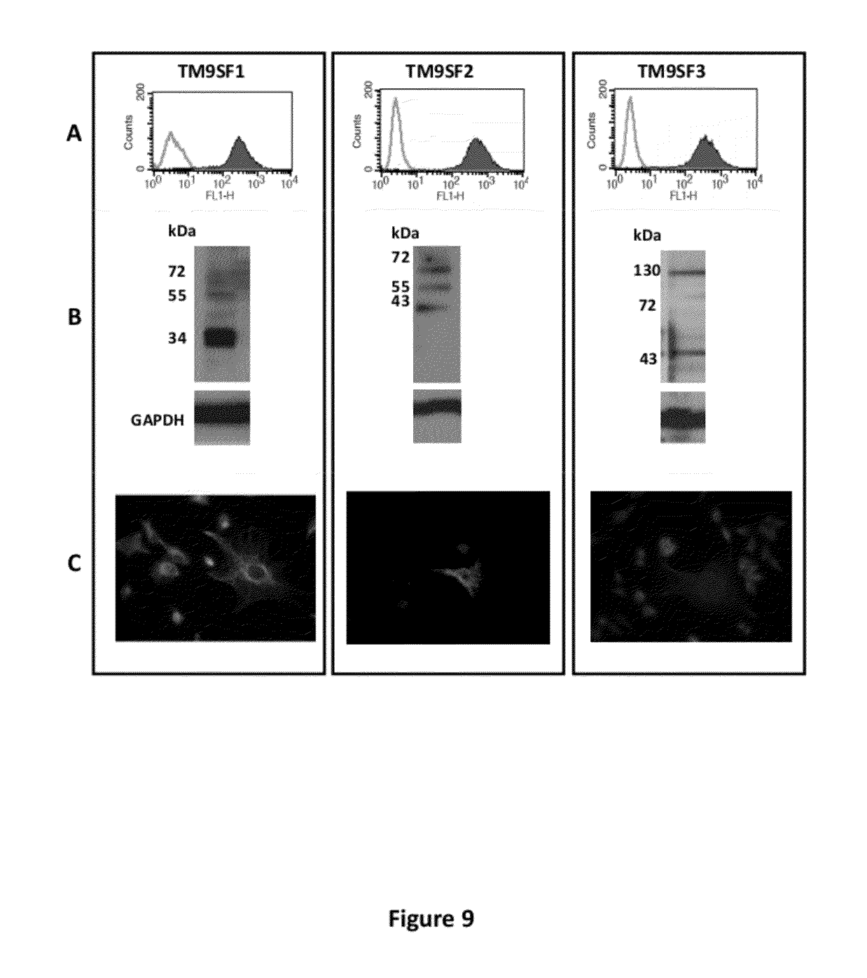

FIG. 9. Expression of TM9SF1, TM9SF2 and TM9SF3 on tumor cells. Expression analysis of anti-TM9SF1 (left) TM9SF2 (middle) and TM9SF3 (Right).
A. FACS-analysis of TM9SF proteins on MM1 cells, Green line: negative control, Purple: TM9SF proteins.
B. Western Blot analysis of Colo1 whole cell lysates immunoblotted with anti TM9SF proteins antibodies. GAPDH was used as housekeeping protein.
C. Immunofluorescence analysis of Colo cells stained TM9SF1 monoclonal antibodies. As negative controls control isotype antibodies were used. These results were obtained on both Colo colon carcinoma and MM1 melanoma and cell lines (not shown).

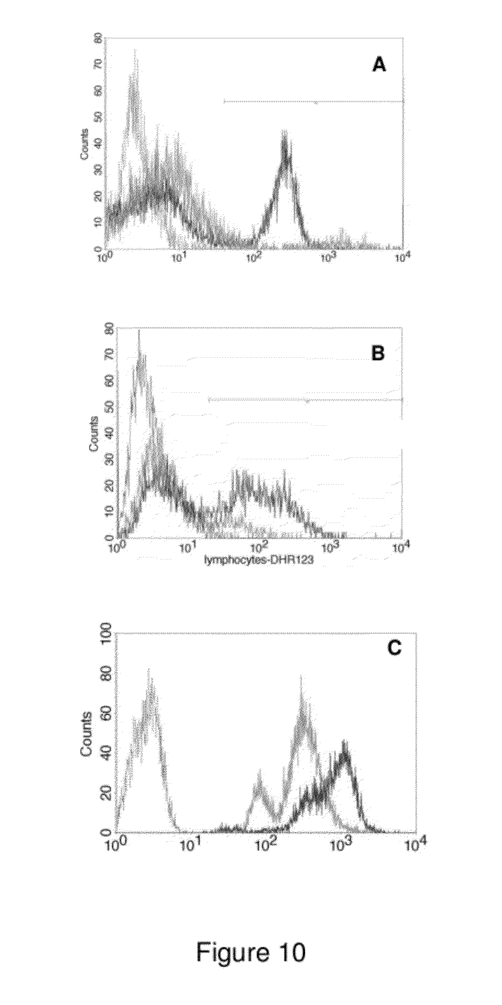

FIG. 10. Functional analysis of TM9SF4/TUCAP-1
A. FACS analysis of phagocytic activity of Scrambled siRNA (SC-siRNA) or TUCAP-1 siRNA (TM9SF4 siRNA) transfected MM2 cells. Gray: unstained control cells; red: negative control Scrambled siRNA transfected cells; green: TM9SF4 siRNA transfected cells.
B. FACS analysis of cannibal activity of SC-siRNA or TM9SF4 siRNA transfected MM2 cells incubated 18 hours with DHR123-stained lymphocytes. Gray: unstained control cells; red: SC-siRNA transfected cells; Green: TM9SF4 siRNA transfected. Cells. To exclude DHR123-stained lymphocytes not ingested with melanoma cells, exclusively melanoma cell fluorescence emission was evaluated.
C. FACS analysis of LysoTracker DND-26 staining of SC-siRNA or TM9SF4 siRNA transfected MM2 cells. Gray: unstained control cells; red: SC-siRNA transfected; Green: TM9SF4 siRNA transfected.

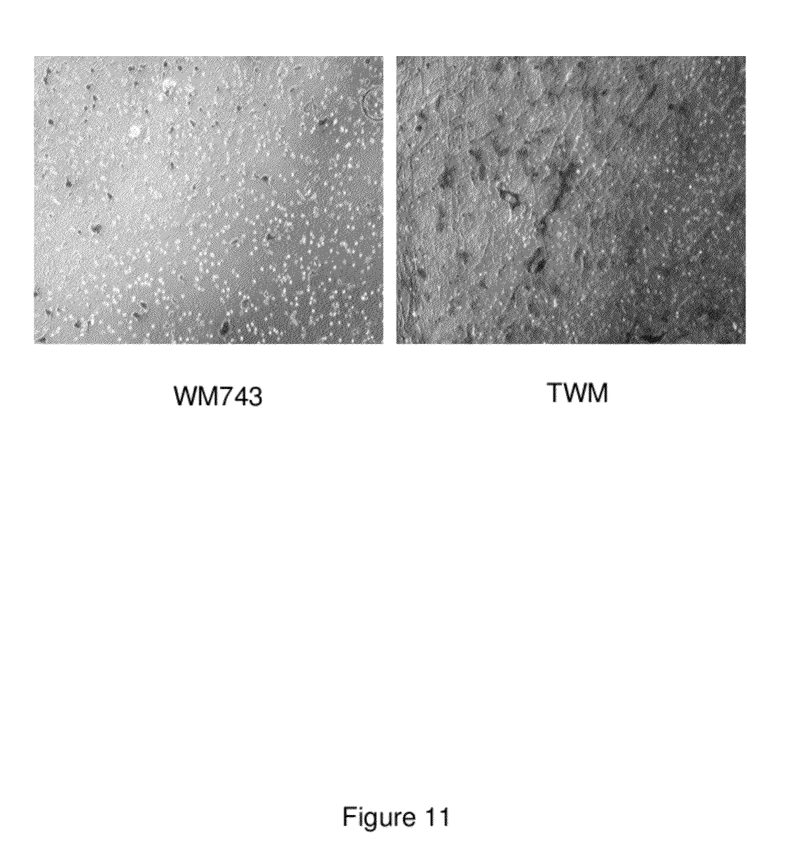

FIG. 11. TUCAP-1-overexpression enhances cell invasion through Matrigel. Phase micrograph of invading WM743 melanoma cells as compared to GFP-Tagged full length TUCAP-1 WM743 melanoma cells (TWM). Invading cells were Fixed in formaldehyde and stained with crystal violet. Picture clearly show that the number of invading cells was significantly higher for TWM, with a mean of 25 cells for untransfected versus a mean of 132 cells for TUCAP 1 transfected TWM.

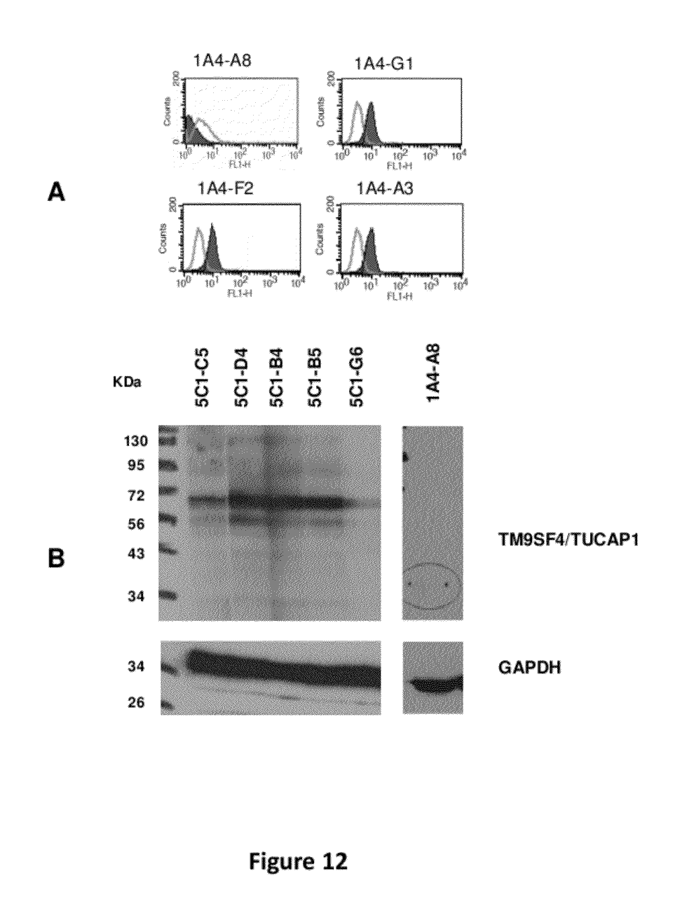

FIG. 12. Examples of applications of anti-TM9SF4/TUCAP-1 monoclonal antibodies.
A. FACS-analysis of MM1 metastatic melanoma cell line for TM9SF4-protein detection with the following clones of anti-TM9SF4 monoclonal antibodies: 1A4-A3, 1A4-A8, 1A4-F2 and 1A4-G1. Green line represents negative control and filled purple represents tested clones.
B. Western Blot analysis of MM1 whole lysates for TM9SF4-protein detection with the following clones of anti-TM9SF4 monoclonal antibodies: 5-C1-C5; 5C1-D4, 5C1-B4, 5C1-C5, 5C1-G6, and 1°4-A8. GADPH was used as a housekeeping protein.

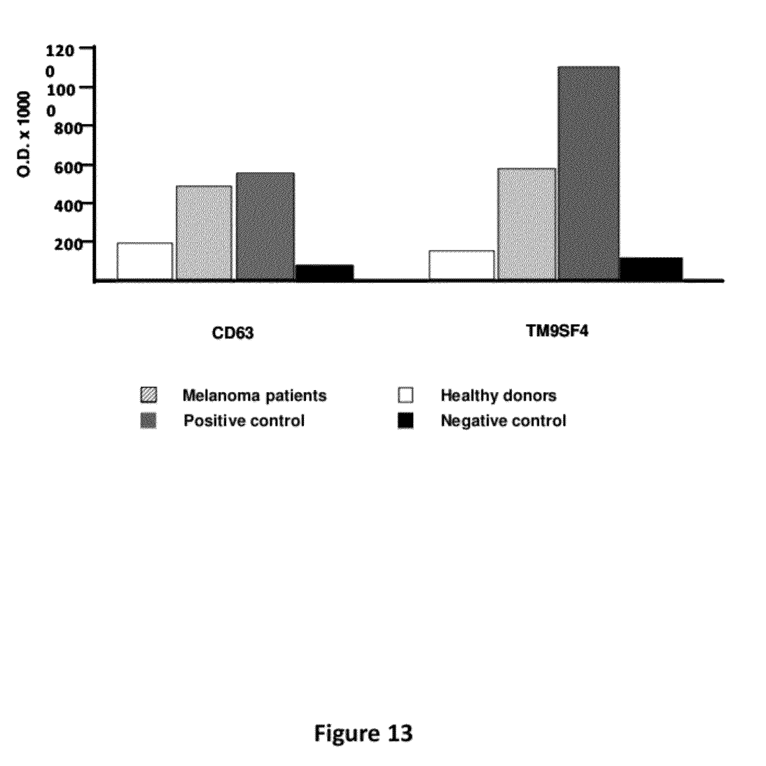

FIG. 13. ExoTest analysis for TM9SF4-expression on exosomes derived from plasma samples.
Exosomes purified from five samples of healthy donors and five samples of melanoma patients were analyzed by ExoTest for the detection of TM9SF4 and CD83. As positive controls the same antigens were detected on 50 μg of exosomes purified from MM1 supernatants. Negative control: Rab5 coated wells plus detecting antibodies (antibodies to TM9SF4 or CD63) and secondary antibody. Exosomes levels are expressed as OD (wavelength 450 nm)×1000.

### DESCRIPTION OF THE INVENTION

### Definitions

Antibody preparation as used in this application includes molecules comprising the antibody or its fragment. Such preparations include chimeric antibodies, humanized and fully human antibodies, conjugates of the antibody or its fragment and a drug molecule or other usefull molecule. The drug molecule can be a medicinal molecule or product approved for cancer treatment or any other therapeutic compound for treatment of a disease related to altered expression of TM9SF-proteins.

TM9SF-Transmembrane 9 Super Family is a very close related family of proteins with a high degree of homology. Proteins belonging to the Super Family include TM9SF1, TM9SF2, TM9SF3, and TM9SF4 (also called TUCAP1).

TM9SF1-protein is encoded by tm9sf1-gene located in chromosome 14 (map 14q11.2) and having nucleic acid sequence according to SEQ ID NO:7. TM9SF1-protein has amino acid sequence according to SEQ ID NO: 8.

TM9SF2-protein is encoded by tm9sf2-gene located in chromosome 13 (map 13q32.3) and having nucleic acid sequence according to SEQ ID NO: 3. TM9SF2-protein has amino acid sequence according to SEQ ID NO: 4.

TM9SF3-protein is encoded by tm9sf3-gene located in chromosome 10 (map 10q24.1) and having nucleic acid sequence according to SEQ ID NO: 5. TM9SF2-protein has amino acid sequence according to SEQ ID NO: 6.

TM9SF4-protein, as used in this application is Human Genome Project—nomenclature and a synonym of TUCAP-1 protein. The protein is encoded by tucap1-gene (tm9sf4-gene) located in chromosome 20q11.21 and having nucleic acid sequence according to SEQ ID NO: 1. TM9SF4-protein has an amino acid sequence according to SEQ ID NO: 2. The structure of the protein is shown in FIG. 1.

TUCAP 1-protein (Tumor Associated Cannibal Protein), as used in this application is a synonym of TM9SF4 (Human Genome Project nomenclature). The protein is encoded by tucap 1-gene (tm9sf4-gene) located in chromosome 20q11.21 and having nucleic acid sequence according to SEQ ID NO:1 TUCAP-protein has an amino acid sequence according to SEQ ID NO:2.

ExoTest™ is a trademarked ELISA-based test that was first described and claimed in the U.S. provisional patent application No. 61/062,528 and subsequent non-provisional patent application Serial Number U.S. 2009/0220944, both of which are incorporated herein by reference. ExoTest platform comprises ELISA plates pre-coated with antibodies against housekeeping exosome proteins (Housekeeping protein stands for the protein ubiquitously expressed on all exosomes in both physiological and pathological conditions) enabling specific capture of exosomes from different biological samples, including cell culture supernatants and human biological fluids. Quantification and characterization of exosomal proteins is subsequently performed by using appropriate detection antibodies against exosome associated antigens that can be either common for all exosomes or cell type- or cell condition specific. By employing different combinations of capture and detection antibodies ExoTest can be customized for assessing multiple antigens in a total exosome population as well as enrichment with cell/tissue specific exosomes from body fluids. The assay provides an immediate readout, namely origin, quantity and molecular composition of isolated exosomes. For the samples of interest RNA (mRNA or miRNA) can be extracted and analysed from captured exosomes.

Exosomes are small endosome-derived vesicles of a size ranging between 30-120 nm and made up of a lipid bilayer that incorporate a characteristic set of proteins, including a large quantity of tetraspanins such as CD9 and CD81, all the known antigen presenting molecules, and several cytosolic proteins. Exosomes are released in normal and pathological conditions, but amount and molecular composition of released exosomes depend on the state of a parent cell.

### DESCRIPTION

TUCAP-1 belongs to the Transmembrane 9 Superfamily (TM9SF), a highly conserved family of proteins characterized by the presence of a large variable extracellular N-terminal domain and nine to ten putative Transmembrane domains. Function and localization of the protein was not described before U.S. Serial Number 2009/0191222 and the corresponding provisional application No. 61/062,453, both of which are incorporated herein by reference, which disclosed that TUCAP1-protein is highly expressed in malignant cells, and that the protein was undetectable on cell lines deriving from primary lesions but was present in malignant melanoma cell lines. Moreover, the protein was shown to be involved in the phagocyte behavior of metastatic melanoma cells, since silencing the gene encoding the proteins strongly inhibited the phagocytic behavior of metastatic cells. FIG. 1 shows the molecular structure of TM9SF4/TUCAP-1-protein. FIG. 3 shows expression of the protein in malignant melanoma cells. FIG. 2 shows the molecular structure of TM9SF 1, 2 and 3 proteins.

In this disclosure the expression of TM9SF proteins on tumor cells is addressed on several tumor model lines. The expression of TM9SF4 protein was previously characterized (U.S. Serial Number 2009/0191222 and 61/062,453, both of which are incorporated herein by reference) on malignant melanoma cells, healthy skin cells, peripheral blood lymphocytes and differentiated macrophages, confirming specific presence of the protein on tumor cells, as shown in FIGS. 3, 4 and 5. The expression of the TM9SF4-protein is also shown on Colo1 (colon carcinoma) cells by FACS and WB (FIG. 7). In addition, the expression of TM9SF4 (TUCAP1) is addressed by RT-PCR in different tumor lines comprising B lymphoma, colon carcinoma, breast carcinoma, osteosarcoma, prostate cancer and ovary cancer (FIG. 8).

The expression of TM9SF1, TM9SF2 and TM9SF3 on tumor cells is characterized on melanoma (MM1) and colon carcinoma (Colo1) cells by FACS, WB and Immunofluorescence analysis (FIG. 9 A-C). All three proteins were found expressed on the model cell lines.

To address exosome association of TM9SF-proteins, in a first set of experiments we used exosome preparations from conditioned culture media of human tumor cell lines to evaluate the expression of TM9SF-proteins on exosomes by FACS and WB. The results showed that all the proteins belonging to the TM9-Superfamily are detectable on exosomes (results not shown). The non-provisional patent application entitled “A method and a kit to detect malignant tumors and to provide a prognosis’ for Francesco Lozupone, Mariantonia Logozzi, Stefano Fais, Antonio Chiesi, and Natasa Zarovni, with an application number to be determined, and filed on the same day as this application discloses an Exotest using TM9SF-antibodies of this application for detection and characterization of exosomes. The application is incorporated herein by reference.

A preferred embodiment of the invention is an antibody which is a whole antibody molecule or fragment thereof that recognizes (or can bind to) specific sequences of TM9SF4/TUCAP-1 protein, which is its antigen. The antibody may be either a polyclonal antibody or a monoclonal antibody. In this embodiment, TUCAP-1 protein is a polypeptide having the amino acid sequence according to SEQ ID NO: 2, and the specific sequences are polypeptides having an amino acid sequence containing deletion, substitution or addition of one or more amino acids as compared to the amino acid sequence of SEQ ID NO: 2 or a fragment thereof. The antibody of the present invention encompasses antibody mutants. An “antibody mutant” is a mutant in which one or more amino acid residues in the antibody have been modified from the original.

Another preferred embodiment of the invention is antibody preparations including molecules comprising the antibody or its fragment. Such preparations include chimeric antibodies, humanized and fully human antibodies, conjugates of the antibody or its fragment and a drug molecule or other useful molecule. The drug molecule can be a medicinal molecule or product approved for cancer treatment or any other therapeutic compound for treatment of a disease related to altered expression of TM9SF-proteins.

A preferred embodiment of the invention are monoclonal antibodies, fragments thereof and antibody preparations that are capable of recognizing specific sequences of TM9SF-proteins, including TM9SF1, TM9SF2, TM9SF3, and TM9SF4 (TUCAP-1).

According to another preferred embodiment of the invention are selected hybridoma cell lines producing the antibodies of this invention.

A preferred embodiment of this invention is eight cell lines that were selected for production of anti-TM9SF4-antibodies. The cell lines are named 1A4-A3, 1A4-A8, 1A4-F2, 1A4-G1, 5C1-B4, 5C1-C5, 5C1-D4, and 5C1-G6 and they are available from Hansabiomed OU (c.f. www.hansabiomed.com).

Further embodiments of the invention are TM9SF-inhibitors. In the case of TM9SF4, such inhibitor molecules may be polynucleotide sequences that are substantially complementary to the sequence of SEQ ID NO: 1 or part of it, and oligonucleotide sequences substantially complementary to a fragment of SEQ ID NO: 1. In case of TM9SF1 such molecules may be polynucleotide sequences that are substantially complementary to the sequence of SEQ ID NO: 7 or part of it, and oligonucleotide sequences substantially complementary to a fragment of SEQ ID NO: 7. In case of TM9SF2 such molecules may be polynucleotide sequences that are substantially complementary to the sequence of SEQ ID NO: 3 or part of it, and oligonucleotide sequences substantially complementary to a fragment of SEQ ID NO: 3. In the case of TM9SF3 such molecules may be polynucleotide sequences that are substantially complementary to the sequence of SEQ ID NO: 5 or part of it, and oligonucleotide sequences substantially complementary to a fragment of SEQ ID NO:5.

According to yet another embodiment of the invention are methods are included for treating cancer in a human patient comprising the step of administering to the patient a therapeutically effective amount of a composition comprising a TM9SF-binding agent conjugated to a chemotherapeutic drug. Antibodies and fragments that specifically bind to TM9SF-protein can be used to treat cancers. The invention includes the use of antibodies and antibody fragments that are fused to other moieties that can have a cytotoxic or immunomodulatory effect on cancer.

Yet another preferred embodiment of this invention is a kit to detect TM9SF from tissue specimens and body fluids of tumor patients as a diagnostic and/or prognostic tool such as a detection kit. Such kit comprises: a) anti-TM9SF antibodies; b) a positive control consisting of the purified TM9SF-proteins, and the necessary buffers.

According to a preferred embodiment such kits also include washing buffer solutions, diluents for the samples of biological fluid to be assayed, a chromogen, a solution of tetramethylbenzidine (TMB), and a stop solution, or another enzymatic substrate solution such as chemilumiscence one for assay development.

Selected hybridomas produce anti-TM9SF monoclonal antibodies according to the invention by using conventional methods such as those for example described in Example 5 below.

Preparation of the specific anti-TM9SF4 mouse monoclonal antibodies from the hybridomas of the present invention is not subject to particular restrictions and can be carried out by conventional methods such as those for example described in Example 5 below. The monoclonal antibodies of this invention may be made by any available method, such as, but not limited to recombinant DNA-technologies or chemical synthesis.

The invention is now described by means of examples, which are not meant to limit the scope of the invention. The scope of the invention is defined by the appended claims.

### Example 1

### Tucap-1-Transcripts and TUCAP-1-Protein are Detectable in Human Malignant Melanoma Cells, but not in Primary Melanoma Cells, in Peripheral Blood Mononuclear Cells or in Healthy Skin Cells

Cell culture. Human primary and metastatic melanoma cell lines were respectively derived from primary or metastatic tumor lesions of patients surgically resected at the Istituto Nazionale dei Tumori, Milan, Italy. All cells employed in the current study were designated by PM (primary melanoma) or MM (metastatic melanoma), followed by a number. Human peripheral blood mononuclear cells (PBMC) were purified by Ficoll-Hypaque (Pharmacia) density gradient of buffy coats from healthy donors. Monocytes were separated from PBMC by using CD14 labeled Miltenyi microbeads according to manufacturer's indications and were left to differentiate for 2 weeks at 37° C. in RPMI 1640 plus 15% FCS. Remaining peripheral blood lymphocytes (PBL), were obtained after CD14 beads mediated monocyte ablation. All the cells were seeded in RPMI 1640 supplemented with 100 IU/mL penicillin, 100 μg/mL streptomycin, 10% FCS in a 5% CO₂ environment at 3TC. (All reagents were purchased from Cambrex).

PCR analysis. Expression of Tucap-1 transcripts was assessed by rt-PCR on several primary and metastatic melanoma cell lines obtained from melanomas of patients surgically resected at Instituto Nazionale Tumori, Milan, as compared to peripheral blood lymphocytes (PBL). Total RNA from the cells was obtained by the RNAzoI (Invitrogen) method and RNA templates were used for RT-PCR amplification. Primers for TUCAP-1 detection were:

(SEQ ID NO: 9)
Forward: tgtgtgaaacaagcgccttc,
and
(SEQ ID NO: 10)
Reverse: atgaggtggacgtagtagt.

These primers amplify a fragment of 349 base pairs.

Primers used to direct TUCAP-1 His-tagged N-terminal domain synthesis were:

(SEQ ID NO: 11)
gaattcatgtgtgaaacaagcgcctt,
and
(SEQ ID NO: 12)
gtcgacagaaaaccagtggatctg.

Primers to detect GAPDH were:

(SEQ ID NO: 13)
Forward: ccatggagaaggctgggg,
and
(SEQ ID NO: 14)
Reverse: caaagttgtcatggatgacc.

TUCAP 1 cloning and expression of TUCAP 1 fusion protein in human melanoma cells: PCR products were cloned into pTopo vector (Invitrogen) and then excised with the appropriate pair of restriction enzymes (EcoRI, SalI) to acquire a single fragment that was subsequently ligated in the pTrcHis2 vector (Invitrogen). The expressed recombinant protein was purified employing Ni NTA agarose resin (Qiagen) following manufacturer's instructions and utilized to immunize mice.

Primers that were used to direct GFP-tagged full length TUCAP-1 were:

(SEQ ID NO: 15)
Forward: gaattcatgtgtgaaacaagcg,
and
(SEQ ID NO: 16)
Reverse: gtcgatgtctatcttcacagcata.

PCR products were cloned into pTopo vector (Invitrogen) and then excised with the appropriate couple of restriction enzymes (EcoRI-SalI) and ligated to acquire a single fragment that subsequently was ligated in the pEGFPN1 vector (Clontech) at the EcoRI and SalI sites to produce the GFP-TUCAP-1 fusion protein. Plasmids encoding the GFP-TUCAP-1 fusion protein were transfected into MM1 and MM2 cells by using the Lipofectamine 2000 transfection kit (Invitrogen) according to the manufacturer's instructions, thus obtaining GFP-TUCAP-1 (GFP-Tuc) MM1 or MM2 cells. The percentage of transfected cells was evaluated by Fluorescence-activated cell sorting analysis.

### Western Blotting and Immunoprecipitation

Bacterial lysates, whole melanoma cell lysates and CCD-1064SK healthy skin fibroblasts (SantaCruz) were resuspended in SDS sample buffer, denaturated by boiling, separated by SDS-PAGE, and analyzed by Western blot. 6×His tagged protein, GFP, TUCAP-1, and GAPDH, were respectively detected with anti6His mAb (Sigma), anti GFP (clone 1 E4 MBL), anti TUCAP-1 mouse serum and antiGAPDH (SantaCruz). TUCAP-1 proteins were immunoprecipitated overnight at 4° C. in the presence of protein A+G-Sepharose beads (Pierce) from precleared cell lysates, by using rabbit anti-TUCAP-1 pAb antibody. Rabbit preimmune serum was used as negative control. Actin was detected with anti actin mAb (Sigma).

In order to characterize tucap-1-gene product, cDNA derived from MM1 cells was cloned in bacterial expression vectors to obtain TUCAP-1 first 265 amino acids fused to a 6-Histidine N-terminal tag (6H-Nt-TUCAP 1). Western blot analysis of purified recombinant protein resulted in a translation product of about 30 kDa absent in control bacterial whole lysates (negative control). Therefore, His-tagged TUCAP-1 recombinant peptide was employed as immunogen to produce anti-TUCAP-1 antibodies in mice. The specificity of the TUCAP-1 antiserum was determined by Western blot analysis of the purified 6H-Nt-TUCAP-1 immunoblotted with anti 6His and TUCAP-1 mouse antisera (FIG. 3B). TUCAP-1 mouse antiserum was further analyzed by Western blot on Triton soluble and Triton insoluble fractions of MM1 cells transfected or not transfected with a GFP-tagged full length TUCAP-1 (GFP-TUCAP-1). The anti-GFP antibody revealed a single specific translation product in the 100 kDa range, while anti-TUCAP-1 antibodies recognized both the GFP-tagged and the endogenous TUCAP-1 corresponding to a 70 kDa protein detectable in both cell lines (FIG. 3C). Interestingly, TUCAP-1 was more represented in the Triton insoluble fractions (GAPDH negative, cytoskeletal proteins enriched fraction), thus supporting the provisional models proposing TUCAP-1 as a transmembrane protein. To further support PCR results, the anti-TUCAP-1 antibodies were blotted in cellular extracts of four metastatic melanoma cells (MM2-MM5), previously analyzed for their cannibal behavior as compared to healthy skin fibroblasts (HSC) and GFP-TUCAP-1 transfected MM2 cells, as a control. TUCAP-1 was exclusively detectable in melanoma cells, while undetectable in skin cells (FIG. 4).

### Example 2

### Immunochemistry of TUCAP-1

Immunochemistry shows TUCAP-1 exclusively in melanoma cells immunocytochemistry and immunohistochemistry. For immunocytochemistry, melanoma cells and macrophages, cultured on glass chamber slides (Falcon), and PBL, cytospun on glass slides, were fixed with 80% methanol 10 minutes at 4° C. and stained for TUCAP-1, TUCAP-1 mouse serum or preimmune control serum. Malignant melanoma and corresponding normal skin tissue from Biomax array slides (Biomax) were immunostained with pre-immune serum, for anti-TUCAP-1 mouse antiserum. Melanoma was also stained for anti-gp100 (Immunotech) while normal skin was also stained for anti-ezrin (Sigma). Proteins were visualized using the peroxidase antiperoxidase method in single staining (Dako) and counterstained with Mayer's hematoxylin.

FIG. 5A-C shows that MM2 cell lines (A), Peripheral blood lymphocytes (B), and in vitro differentiated Macrophages (C), were negative for mouse preimmune serum. Consistent with PCR results malignant melanoma cultured cells showed clear positive staining for TUCAP-1 (FIG. 5 D) while PBL (FIG. 5E) and macrophages (FIG. 5F) were negative for TUCAP1 staining. Immunohistochemical analysis of malignant melanoma tissues as compared to healthy skin suggested that TUCAP-1 was detectable only in melanoma tissues (FIG. 5H) while undetectable in healthy skin (5K). As positive control markers for melanoma and normal skin GP100 (FIG. 5I), and ezrin (FIG. 5L) were used respectively. Pre-immune mouse serum staining was always negative in both tissues (FIGS. 5G, 5J). These results provide clear evidence that TUCAP-1 was exclusively detectable in melanoma cells and thus support the results of Example 1.

### Example 3

### Subcellular Localization of TUCAP-1

Further experiments were performed to analyze the intracellular localization of TUCAP 1.

Cell compartment fractionation cells were harvested and processed according to Qproteome plasma membrane kit protocol (Quiagen) in order to obtain non denatured fractions of cellular compartments corresponding to purified plasma membranes and cytosol. The latter fractions were then precipitated with acetone and resuspended in immunoprecipitation buffer B (0.1% SDS, 1% NP40, 0.5% sodium cholate) in order to be subjected to immunoprecipitation with rabbit anti TUCAP-1. Residual pellet from cellular compartment fractionation, containing intact cells and organelles, was deprived of the former through centrifugation and subjected to Triton X-100 extraction in order to obtain soluble and insoluble fractions which were immunoprecipitated with rabbit anti TUCAP-1. Following electrophoresis of samples, the nitrocellulose was blotted with mouse anti-TUCAP-1.

Immunofluorescence analyses MM2 cells were seeded on cover glass placed in 60-mm Petri dishes. Cells were fixed with 2% paraformaldehyde and permeabilized (Triton X-100 (0.1%) or 24 hous. For TUCAP 1 and Rab5 double staining cells were labeled with mouse anti-TUCAP-1 serum and rabbit anti-Rab5 (SantaCruz) and respectively revealed with Alexa Fluor 488-conjugated anti-mouse IgG and anti-rabbit Alexa Fluor 594-conjugated IgG (Molecular Probes). For TUCAP-1 and Lamp-1 detection cells were labeled with rabbit anti-TUCAP-1 pAb and mouse anti Lamp-1 Mab, (BD Pharmingen) respectively, stained with Alexa Fluor 594-conjugated anti-rabbit IgG and Alexa Fluor 488-conjugated anti-mouse IgG. TUCAP-1 and mitochondria were detected by staining TUCAP-1 with anti-TUCAP-1 mouse pAb and labeled with Alexa Fluor 488-conjugated anti-mouse IgG, while mitochondria were labeled with Mithotracker Red (Invitrogen). After washings, all samples were mounted with glycerol:PBS (2:1) and observed with a Leica DM 2500 fluorescence microscope. Images were recorded with a Spot Insight digital camera (Delta Sistemi) equipped with IAS 8.2 system of image analysis (Delta Sistemi).

MM2 whole cell lysates were immunoprecipitated with anti-TUCAP-1 antibodies and various subcellular fractions were separated and analyzed by Western blot. The results revealed that TUCAP-1 was mainly recovered in fractions enriched for cellular organelles, while undetectable in sytosolic and plasma membrane fractions. In order to identify subcellular localization of TUCAP-1, MM2 cells were double stained for TUCAP-1, and either for the early endosomal markers Rab5, or for the component of late endosomes and lysosomes Lamp-1, or the mitochondrial marker Mitotracker™. Fluorescence microscopy analysis showed that TUCAP-1 co localized with both Rab5 and EEA-1), while it did not co-localize with either Lamp-1, Mitotracker™ or Hoechst stained nuclei (Results shown in U.S. Serial Number 2009/0191222 are incorporated herein by reference).

Moreover, FIG. 6 shows the double staining fluorescence on same cells co-cultured with living lymphocytes. TUCAP-1 is detectable exclusively on the surface of melanoma cells, while lymphocytes are completely unstained (FIG. 6A). Again, TUCAP-1 co-localizes with the primary endosome marker EEA-1 (FIG. 6B) confirming the expression of this protein on early endosomes.

### Example 4

### Polyclonal Antibodies to TM9SF-Proteins

In order to produce polyclonal antibodies to TM9SF4 (TUCAP-1), cDNA from MM1 cells was cloned in bacterial expression vectors to obtain TUCAP-1 amino acids 18-279 (SEQ ID NO: 17) fused to a 10-Histidine N-terminal tag (SEQ ID NO: 27). Purified recombinant peptide was used to produce anti-TUCAP-1 antibodies in mice. The anti-TUCAP-1 antibodies recognized immunogen, GFP-tagged full length protein as positive control as well as endogenous TUCAP-1 protein.

Polyclonal antibodies were also generated by immunizing a rabbit with a purified peptide fragment having an amino acid sequence according to SEQ ID NO: 18. The antibodies generated were able to recognize human TUCAP-1 protein by binding to a peptide fragment that consists of amino acids 221-235 of SEQ ID NO: 1. Polyclonal antibodies are also obtained by immunizing a goat and a donkey.

Polyclonal antibodies against TM9SF1 were produced similarly using amino acids 90-215 of SEQ ID NO:8 (SEQ ID NO: 19) fused to a 10-Histidine N-terminal tag (SEQ ID NO:27).

Polyclonal antibodies against TM9SF2 were produced similarly using amino acids 106-271 of SEQ ID NO:4 (SEQ ID NO:20) fused to a 10-Histidine N-terminal tag (SEQ ID NO:27).

Polyclonal antibodies against TM9SF3 were produced similarly using amino acids 29-222 of SEQ ID NO: 6 (SEQ ID NO:21) fused to a 10-Histidine N-terminal tag (SEQ ID NO:27).

### Example 5

### Monoclonal Antibodies for TM9SF-Proteins and Hybridoma Cell Lines Producing the Antibodies

In order to produce monoclonal antibodies for TM9SF4/TUCAP-1, mice were immunized with a peptide fragment having amino acid sequence according to SEQ ID NO:17 (amino acids 18-279 of SEQ ID NO:2). Selected hybridoma clones were generated by using spleen cells of selected mice. Briefly B-cells deriving from spleen of immunized mice were fused with a myeloma tumor cell line specifically selected for hybridoma production. The derived fused (hybrid) cells that can grow indefinitely in culture with consequent production large amounts of the desired antibodies. Hybridoma production was performed according to standard protocols as described below. After screening the selected hybridomas, the hybridomas were cloned and grown to large-scale for antibody production. Eight positive hybridomas that efficiently produce anti-TM9SF4-antibodies were selected. The antibodies have been used in laboratory experiments such as Western Blot, immuno-precipitation, FACS analysis, immunofluorescence and immunohisto- and immunocyto-chemical analysis of human tissues and cultured cells, in preclinical and clinical studies, as a part of tumor diagnosis and prognosis tools, such as detection kit and in cancer treatements. The monoclonal antibodies produced bind to conformational or linear epitopes of TUCAP-1 protein amino acids 18-279 of SEQ ID NO: 2. The antibodies also bind to TUCAP-1 protein of mouse, rat, cat, dog, and sheep origin.

Monoclonal antibodies against TM9SF1 were produced similarly using SEQ ID NO: 19, i.e. amino acids 90-215 of SEQ ID NO:8. The monoclonal antibodies were able to detect TM9SF1 protein (SEQ ID NO: 8) and TM9SF1-isoforms of SEQ ID NO: 39, 40, 41, 41 and 42.

Monoclonal antibodies against TM9SF2 were produced similarly using SEQ ID NO:20, i.e. amino acids 106-271 of SEQ ID NO:4. The monoclonal antibodies were able to detect TM9SF2 protein (SEQ ID NO:4).

Monoclonal antibodies against TM9SF3 were produced similarly using SEQ ID NO: 21, i.e. amino acids 29-222 of SEQ ID NO: 6. The monoclonal antibodies were able to detect TM9SF3 protein SEQ ID NO:6).

The procedure for creating hybridomas and monoclonal antibodies is described here in more detail.

### Selection of Immunogen and Cloning

TM9SF4/TUCAP1 analysis of secondary structure revealed the presence of a large hydrophylic N-terminal domain followed by nine transmembrane domains (FIG. 1). This analysis, together with Blast analysis of identity percentage with the other proteins of the same superfamily, allowed the identification and selection of the TM9SF4/TUCAP1 (Gen Bank: CAQ09924.1) N-terminal domain corresponding to aa 18-279 (SEQ ID NO: 17).

FIG. 2 shows secondary structure of TM9SF1, 2 and 3. Analysis of the structure together with Blast analysis led to identify the following domains:

For TM9SF4/TUCAP-1 a nucleotide sequence (SEQ ID NO: 22) was amplified from human cDNA using the following oligonucleotides as primers:

Forward primer TucapFOR (21 nt; Tm: 57.5° C.)
(SEQ ID NO: 23)
atgtgtgaaacaagcgccttc.
Reverse primer TucapREV (22 nt; Tm: 58.4° C.)
(SEQ ID NO: 24)
gtggatctggacgtcactcatg.

The specific sequences required for cloning steps were added with following oligonucleotides:

Forward primer with LIC-extention: TucapFORlic
(SEQ ID NO: 25)
caccaccacggcgtcatgtgtgaaacaagcgccttc.
Reverse primer with LIC-extension: TucapREVlic
(SEQ ID NO: 26)
cgagcgaaggcgtcagattagtggatctggacgtcactcatg.

Underlined sequences allow the application of LIC technology (Ligation Independent Cloning; Aslandis and de Jong, 1990; Haun et al., 1992) to clone amplified insert in p2N, an expression vector used to express target sequenced as histidine fusion proteind; p2N encode for the following N-terminal 18 aminoacids long tag: MGSDKIHHHHHHHHHHGV (SEQ ID NO:27).

For TM9SF1 the nucleotide sequence (SEQ ID NO: 28) was amplified from human cDNA using the following oligonucleotides as primers:

(SEQ ID NO: 29)
Forward primer: gagtctttgtatgagatccgc
(SEQ ID NO: 30)
Reverse primer acgctccactgaagtctc

For TM9SF2 the nucleotide sequence (SEQ ID NO: 31) was amplified from human cDNA using the following oligonucleotides as primers:

(SEQ ID NO: 32)
Forward primer: gaaccttcaccatataagtttacgtttaa
(SEQ ID NO: 33)
Reverse primer gtaagtataggcaatttttatctcccc

For TM9SF3 the nucleotide sequence (SEQ ID NO: 34) was amplified from human cDNA using the following oligonucleotides as primers:

(SEQ ID NO: 35)
Forward primer: gacgagcacgaacacacgtatc
(SEQ ID NO: 36)
Reverse primer atgaatccgatgttgaaaaaag

### Recombinant Protein Production

For recombinant protein production phase, plasmid was used to transform PBM (Primm), a proprietary expression strain grown and induced by autoinduction method (Studer 2005).

After the induction, cells were harvested and broken with enzymatic method. His-TM9SF1, His-TM9SF2, His-TM9SF3 and His-TUCAP1 in each case respectively accumulated in inclusion bodies, solubilized with Guanidine 8M and then purified on Nichel-sepharose column. Purified recombinant protein domains were obtained in denaturing buffer (Urea 6M).

### Hybridoma

### Immunization

BALB/c mice were subcutaneolsly immunized with 10 μg of recombinant protein his-tag TM9SF4/TUCAP1, his-tag TM9SF, his-tag TM9SF2 or his-tag TM9SF3, respectively. The antigen (10 μg) was emulsified in Freund's Complete Adjuvant (CFA) and subcutaneously injected in BALB/c mice. A booster injection of antigen emulsified in Freund's Incomplete Adjuvant (IFA) was administered at 21, 28 days after immunization with antigen/CFA emulsion. At day 35 a serum sample bleed from each mice was tested for antibody concentration with ELISA assay. The last boost pre-fusion was administered 4 days before the final sacrifice to proceed with spleen fusion.

### Splenocyte Preparation

The spleen of immunized mice was harvested in aseptic conditions, transferred to 60 mm-diameter plate containing 3 ml of Complete RPMI-serum free medium and disaggregated into a single-cell suspension by passage through a cell strainer with 70 μm porosity.

The suspension was transferred to 50 ml conical centrifuge tube and washed 3 times with Complete RPMI-serum free medium.

### Myeloma Cell Preparation

The Sp2/0-Ag14 myeloma cells (Health Protection Agency Culture Collection, UK) were transferred from tissue culture flask to 50 ml conical centrifuge tube and washed 3 times with Complete RPMI-serum free medium.

### Fusion

Myeloma cells and splenocytes were mixed in a ratio of 2:1 in a 50 ml conical centrifuge tube. The tube was filled with Complete RPMI-serum free medium and centrifuged 5 min to 500×G. 1 ml of pre-warmed PEG 50% was added drop-by-drop to mixed cells pellet over 1 minute, stirring after each drop and for an additional minute. The fusion was performed at 37° C. 2 ml of pre-warmed Complete RPMI-serum free medium was added drop-by-drop to mixed cells pellet over 2 minute, stirring after each drop. 7 ml of pre-warmed Complete RPMI-serum free medium was added drop-by-drop to mixed cells pellet over 3 minutes, stirring after each drop. The cell suspension was centrifuged 5 min to 500×G. The pellet was resuspended at the concentration of 2.5×10⁶ cell/ml with Complete RPMI-15% FBS/Hepes/Pyruvate medium and the cells suspension was dispensed in 96-well plate (100 μl/well). The plates were incubated at 37° C. and 5% CO₂ conditions.

### Hybridoma Selection

After 1 day of incubation 100 μl of Complete RPMI-15% FBS/Hepes/Pyruvate/HAT medium were added to each well. On days 2, 3, 6, 8, 10 100 μl of supernatant medium were aspired from each well and 100 μl of selecting medium containing HAT (Hypoxanthine, aminopterine, thymidine) was added to each well. The procedure was repeated at day 14, but the well was filled with Complete Medium containing HT (Hypoxanthine, thymidine) instead of HAT. On day 14, 50 μl of hybridoma growing supernatant was tested in ELISA assay.

### Cloning by Limiting Dilution:

The hydridoma cells solution was dispensed in a 96 well-plate to a concentration of 1 cell/well and 0.3 cell/well. After 5/6 days the wells were inspected for monoclonality with inverted microscope.

Around 14-15 days from seeding the supernatant of monoclones was screened with ELISA assay.

### Hybridoma Sub-Isotyping

### Application of Capture Antibody

The Stock Plate Coating Solution was diluted with distilled water, using 9.0 ml distilled water with 1.0 ml coating solution for each plate to be coated. 100 μl of Goat Anti-Mouse Igs (Plate Coating Reagent) was added to the 10 ml of Plate Coating Solution. 100 μl of this plate coating mixture was added to each well of a 96-well EIA plate.

### Application of Supernatants

PBS concentrate was diluted by using 5 ml concentrate for each 100 ml of buffer. For every litre of buffer 500 μl of surfactant was added to reduce non-specific binding. The coated plate(s) were removed from refrigeration, the contents was shaken out into a sink and pat dry on a clean towel. The plate(s) were washed with PBS-surfactant using a gentle stream from the squeeze bottle and each well was filled. The washing step was repeated twice. The blocking serum was diluted in ratio of 1:4 with 1×PBS. 200 μl of diluted blocking serum was added to each well. The plates were incubated at room temperature for 1 hour and washed with PBS-surfactant and pat dry with a clean towel. 50 μl of each hybridoma supernatant was added to one column of 8 wells. The plates were incubated at room temperature for 1 hour.

### Application of Sub-isotyping Antisera

The contents of the incubated plates were shaken out and pat dry. The plates were washed with water, saline, or PBS and pat dry. The washing step was repeated twice. Two drops from each of the typing antisera bottles was added to a different well for each hybridoma tested. 100 μl of PBS-surfactant was added to any wells that do not receive antiserum. These wells are the negative controls. The plates were incubated at room temperature for 1 hour.

### Application of Conjugate

Peroxidase conjugate was deluted in ration of 1:4,000 with PBS. The contents of the antisera incubated plates were shaken out and patted dry. The plates were washed with saline or PBS and pat dry. Washing-step was repeated twice. 100 μl of diluted conjugate was added to each well/ The plates were incubated at room temperature for 1 h and the contents of the plates were shaken out and patted dry. The plates were washed with saline or PBS and patted dry. The washing step was repeated twice.

### Application of Substrate:

The ready-to-use TMB substrate reagent is stable at 4° C. and produces a blue color that can be read at 655 nm. Addition of acid as a stop solution enhances sensitivity 2-4 times and produces a yellow color that can be read at 450 nm. For best results, we removed the amount needed and transfered to a clean container before adding to plates.

100 μl of TMB substrate reagent was added to each well. Color development was complete in 5-10 min, and positive wells developed a bright blue color; negative wells retained a clear to faint blue color. Readings were done at 655 nm. Color development was stopped by adding 50 μl of 1 M phosphoric acid to each well and readings were done at 450 nm.

After cloning by limiting dilution, for the monoclonal anti-TUCAP-1, a larger number of final clones were isolated, approx. 40 sub-clones which have been further investigated with different experiments (western blot analysis, FACS experiments, and IF (results not shown) to identify the best clones producing the antibodies. As a result eight hybridoma cell lines were selected for efficient production of antibodies. The cell lines were named as 1A4-A3, 1A4-A8, 1A4-F2, 1A4-G1, 5C1-B4, 5C1-C5, 5C1-D4, and 5C1-G6. The antibodies were able to detect TM9SF4/TUCAP protein.

### Example 6

### Method for Determining the Levels of TM9SF4 in Patients' Plasma by Using Mouse Anti-TM9SF Antibodies of this Invention

96-well ELISA plates (Nunc MaxiSorp 446612) were coated with 100 μl of a coating buffer solution (15 mM carbonate buffer, pH 9.6) containing purified rabbit TM9SF4 polyclonal antibody 4 μg/ml and incubated overnight at 4° C.; the plates were washed 3 times with 300 μl/well of a washing buffer solution (PBS+0.1% Tween 20) and then 300 μl of washing buffer with 5% of milk powder were added to block non-specific binding sites; after 1 hour at room temperature, the plates were washed 3 times with washing buffer; in duplicate, 50 μl of standard recombinant human TM9SF4 Hydrophilic domain (from 50 pg/ml to 1.5 ng/ml) and samples of the plasma under test diluted in PBS 2% BSA were placed in each well and the plates were incubated for 2 hours at 37° C. Plates were washed 3 times with washing buffer and 100 μl of 25 ng/ml of mouse anti-TM9SF4 monoclonal antibody diluited in PBS 2% BSA were added to each well; the plates were incubated for 2 hours at 37° C. and then washed 5 times with 300 μl of washing buffer; 100 μl of horseradish peroxidase-conjugated anti-mouse secondary antibody (RPN 4401Amersham Copenhagen, Denmark) diluted 1:8000 were added to each well and the plates were incubated for 1 hour at room temperature; after the plates had been washed 3 times with washing buffer, 100 μl of a substrate solution of TMB (tetramethylbenzidine) was added to each well. The plates were incubated for 5 minutes at room temperature; 50 μl of stop solution (H₂SO₄, 1M) was added to each well; the absorbance at 405 nm was read within 30 minutes from the stopping of the reaction.

### Example 7

### A Kit for Determining TM9SF Levels with the Antibodies of this Invention

A kit for determining the levels of TM9SF proteins in human biological fluids comprises: 1. 96 wells Microplate coated with rabbit anti-TM9SF1-4 antibodies. 2. Mouse anti-TM9SF4 IgG in phosphate buffer solution. 3. Horseradish peroxidase-conjugated anti-rabbit secondary antibody in phosphate buffer solution. 4. Standards: purified recombinant TM9SF1-4 proteins at 2.5, 1.25, 0.6, 0.3 and 0.1 ng/ml in buffer solution. 5. Washing buffer solution: phosphate buffer saline (PBS) solution. 6. Diluent (to dilute the human biological fluid under test): 1% bovine serum albumin and 0.19% K3-EDTA in phosphate buffer saline solution. 7. Substrate: 0.26 mg/ml tetramethylbenzidine and 0.01% H₂O₂ stabilised in 0.05 mol/l citrate buffer (pH 3.8). 8. Stop solution: 1M H₂SO₄.

### Example 8

### ExoTest™ Analysis for TM9SF4 Expression on Exosomes Derived from Plasma Samples by Using the Monoclonal Antibodies of this Invention

ExoTest™ analysis of TM9SF4: Basic Exotest™ has been described in US nonprovisional Serial Number 2009/0220944 and in corresponding provisional application 61/062,528, both of which are incorporated herein by reference. Improved Exotest assay is also described in non provisional patent application, application number to be determined, entitled “A method and a kit to detect malignant tumors and to provide a prognosis” for Francesco Lozupone, Mariantonia Logozzi, Stefano Fais, Antonio Chiesi and Natasa Zarovni, filed on the same day as this application, and incorporated herein by reference. Briefly, exosomes purified as described before, were added into anti Rab-5 rabbit pAbs coated ninty-six well-plates (HBM) and incubated overnight at 37° C. After washings with PBS, mouse anti-TM9SF4 antibody produced by a hybridoma cell line selected from cell lines A4-A3, 1A4-A8, 1A4-F2, 1A4-G1, 5C1-B4, 5C1-C5, 5C1-D4, and 5C1-G6, or mouse anti CD63 and CD81 (Pharmingen) antibodies were added as detection antibodies. After washings PBS, the plate was incubated with HRP-conjugated anti-mouse-peroxidase secondary antibody (Pierce) and the reaction was developed with POD (Roche), blocked with 1N H₂SO₄. As negative control, Rab5 coated wells incubated with detecting antibodies followed by secondary antibodies, was used. Optical densities were recorded with an ELISA reader by using a 450 nm filter (Biorad).

Exosomes were purified from the plasma of three different melanoma patients (affected by advance disease stage III-IV) and three healthy donors and were then subjected to ExoTest for TM9SF4 and CD63 detection. Negative control: Rab5 coated wells plus detecting antibodies (antibodies to TM9SF4 or CD63) and secondary antibody. Exosomal proteins levels are expressed as OD (wavelength 450 nm)×1000. Quantification of exosomes based on TM9SF4 expression by ExoTest™ is shown in FIG. 13 that clearly shows that i) TM9SF monoclonal antibodies according to this disclosure have a higher sensitivity for the detection of tumor exosomes when compared with CD63; ii) TM9SF4 values of obtained exosome samples of healthy donors plasma are comparable to negative controls. This suggests that TM9SF4 is associated with exosomes in melanoma patients, and quantification of plasma exosomes bearing this protein is a useful tumor marker. Accordingly the monoclonal antibodies of this invention are useful tools in detecting TM9SF4-protein from samples of patients suspected to have malignant tumors.

### Example 9

### FACS Analysis of TM9SF4 Expression in MM1 Metastatic Melanoma Cells by Using the Antibodies of this Invention

Four different clones of anti-TM9SF4 monoclonal antibodies (1A4-A3; 1A4-A8; 1A4-F2; 1A4-G1) of this invention were tested for their applicability for FACS detection of TM9SF4-protein. Experiments were performed on MM1 melanoma and Colo colon carcinoma cell lines (not shown). As shown in FIG. 12 A all motherclones were able to detect the protein. As negative controls mouse immunoglobulins and secondary antibody were used.

### Example 10

### Western Blot Analysis of TM9SF4 Expression in MM1 Cells

Six different clones of anti-TM9SF4 monoclonal antibodies (5C1-C5; 5C1-D4; 5C1-B4; 5C1-C5; 5C1-G6; 1A4-A8) were tested for their applicability for WB detection of TM9SF4 protein. FIG. 12B show the results obtained by immunoblotting MM1 and Colo whole lysates (not shown) with the 6 clones. All clones tested were able to recognize the protein detecting a band of about 70 kDa, or of about 40 KDa (Clone 1A4-A8). All lanes were immunoblotted with GAPDH as housekeeping protein.

### Example 11

### Tucap-1 Silencing Inhibits Phagocytic Behaviour

The role of TUCAP-1 protein in human metastatic melanoma cells was evaluated by inhibiting its expression trough Tucap-1 silencing.

The following StealthR RNAi duplexes (Invitrogen) were used for tucap-1 silencing:

(SEQ ID NO: 37)
gagugacguccagauccacugguuu,,
and
(SEQ ID NO: 38)
aaaccaguggaucuggacgucacuc.

Duplexes were missing test and annealed according to the manufacturer's instructions. As a negative control Stealth RNAi Negative control medium GC duplexes (Invitrogen) was used. Melanoma cells were transfected using Lipofectamine RNAiMAX reagent (Invitrogen) according to the manufacturer's instructions. Briefly the day before transfection, melanoma cells were seeded in six-well plates (1×10⁵ per well), and after 24 hours, cells were transfected with 30 pmol of siRNA per well. 48 hours after transfection, cells were analyzed for TUCAP-1 expression by FACS analysis.

Three different phagocytic metastatic cell lines were transfected with small interfering RNA to Tucap-1 (Tucap-1 siRNA), or transfected with an unrelevant siRNA oligo (SC-siRNA). Results shown in US Serial number 2009/0191222 are incorporated herein by reference. Here, FIG. 8 shows FACS analysis of TUCAP-1 expression on untransfected MM2 cells and Scrambled siRNA or TUCAP-1 siRNA transfected MM2 cells 48 hours after transfection. Similar results were obtained in scrambled and TUCAP-1 silenced MM3 cells (not shown). This confirmed an effective knockdown of TUCAP-1.

To assess the phagocytic activity of these cells, we first measured the ability of untransfected, SC-siRNA transfected, or Tucap-1 silenced melanoma cell lines to ingest stained yeast cells or living lymphocytes. 48 hours after transfection, SC-siRNA or TUCAP-1 siRNA transfected MM2 and MM3 melanoma cells were incubated at 3TC with FITC stained Saccaromyces yeasts FITC (1:60), or 10 uMol dihydrorhodamine 123 (DHR123) (Molecular Probes) stained living lymphocytes (1:10). Phagocytosis/cannibalism was measured after 4 hours by washing away the excess lymphocytes or yeast cells with PBS and adding a PBS solution containing trypisn (1.5 g/L) EDTA (0.44 g/L). After washings, melanoma cells were harvested and analyzed on a cytometer equipped with a 488-nm argon laser. At least 10,000 venets were acquired and analyzed by a Macintosh computer using CellQuest software (Becton Dickinson). Melanoma cells that appeared fluorescent in green were considered as phagocytic/cannibal. The results showed that the TUCAP-1 knocking-down markedly inhibited both the phagocytic and the cannibal activity of melanoma cells (FIGS. 10 A and B, Table 2), proving that TUCAP-1 plays a key role in the cannibal behavior of metastatic human melanomas and the protein thus can be used as a marker of malignancy.

TABLE 2
Role of TUCAP-1 in phagocytosis/cannibalism. Phagocytotic/Cannibal
activity of scrambled siRNA transfected (SC-siRNA) and Tucap-1-
silenced (Tucap-1 siRNA) MM3 and MM3 metastatic melanoma cells
against FITC stained yeasts and DHR123 stained live lymphocytes.
The phagocytic activity was expressed as % of phagocytic cells.
Numbers re mean ± s.d. of 5 different experiments.

| | |
|---|---|
| Yeasts | Live lymphocytes |

| | | TUCAP | | TUCAP |
|---|---|---|---|---|
| | SC-siRNA | 1-siRNA | SC-siRNA | 1-siRNA |
| MM2 | 37 ± 1 | 4 ± 2 | 45 ± 5 | 17 ± 9 |
| MM3 | 43 ± 14 | 14 ± 12 | 40 ± 9 | 9 ± 7 |

### Example 12

### TUCAP-1 has a Role in Regulating Acidification of Endosomal Vesicles

Scrambled siRNA transfected and TUCAP-1 silenced MM2 and MM3 cells were stained with 1 mu.M LysoTracker probe (Molecular Probes) for 30 minutes at 3T C and immediately analyzed by a cytometer. Comparisons among different melanoma cell lines were conducted by CellQuest software using the median values of fluorescence intensity histograms.

Based on the result that TUCAP-1 localizes on Rab5 bearing endosomes, we tested a hypothesis that TUCAP-1 protein may have a role in the pH regulation of phago/endosomal compartments of malignant tumor cells. To verify this hypothesis, control SC-RNAi and TUCAP-1-siRNA transfected cells were stained with the acidotropic probe LysoTracker green and analyzed by flow symmetry. Tucap-1-gene silencing induced appearance of less acidic vesicle within melanoma cells, as compared to SC-RNA transfected control cells (FIG. 10C). These experiments support the hypothesis that TUCAP-1 has a role in regulating acidification of internal vesicles, such as early endosomes.

### Example 13

### TUCAP-1 Involvement in Early Phases of Metastatic Process

Ongoing experiments based on using TUCAP-1 overexpressing cells suggest that this protein is involved in tumor cell invasiveness during early phases of metastatic process. Cell invasion capability of these cells is assayed by using Matrigel invasion chambers (Becton-Dickenson, Bedford, Mass., USA). Briefly, untransfected WM743 or GFP-Tagged full length TUCAP-1 WM743 melanoma cells (TWM) were resunspended in serum free medium and loaded into the top chamber, while in the bottom chamber was placed in medium added with 10% FCS as a chemoattractant. Cells were incubated at 3° C. in a humidified atmosphere and allowed to migrate through the chemotaxis chamber for 48 hours. After incubation, the cells remaining at the upper surface were completely removed using a cotton carrier. The migrated cells on the bottom of chemotaxis chamber were stained with crystal violet. Invading cells were counted microscopically (40×) in four different fields per filter. FIG. 11 shows the lower side of transwell membrane, clearly indicating that the number of invading cells was significantly higher for TWM, with a mean of 25 cells for untransfected versus a mean of 132 cells for TUCAP 1 transfected TWM cells.

### Example 14

### TUCAP-1 Involvement in Cisplatin Resistance of Melanoma Cells

Several publications show the role of proteins involved in ion trafficking and the role of endo-lysosmal compartment in drug sequestering, inactivation and extrusion as mechanisms of drug resistance. TUCAP 1 expression in early endosomes and its involvement in pH regulation of endosomal vesicles (as shown in above examples) led us to hypothesize a role for this protein in drug resistance of cancer cell. To prove this, MM2 melanoma cells, highly expressing TUCAP-1, were pretreated with Scrambled (SC-siRNA) or Tucap-1 si-RNA for 48 hours (as shown in the previous examples), and after transfection cells were treated with 2 uM cisplatin. 48 hours after cisplatin induced cytotoxicity was evaluated by FACS analysis of early (annexin-V single positive) and late (PI/Annexin V double positive) apoptosis. Tucap-1 silencing markedly increased cytotoxic effects of cisplatin as compared to Scrambled-si-RNA treated WM743 cells that behaved as the untransfected control cells. With a mean of 63% of live cells in control transfected cells versus a mean of 37% in TUCAP-1 silenced cells.

This set of experiments proves that TUCAP-1 is involved in drug resistance of TUCAP-1 over expressing cells and that tucap-1 silencing is a promising method to inhibit phagocytotic character of tumor cells and increase the effect of traditional antitumor treatments.

According to proposed functions of TM9SF4-protein in determining a malignant tumor phenotype, as shown in Examples 8-14, monoclonal antibodies of this invention can be used to interfere with pathways promoting tumor cell invasiveness. Whether TUCAP-1 functions as a membrane receptor or an ion channel, mediating thus intracellular signalling and/or tumor microenvironment, blocking of its activity could arrest the malignant evolution of a tumor and increase the efficiency of traditionally employed anti-tumor therapies. The expression profile of the protein so far confined to tumor cells and scarce or absent on normal adult cells or tissues would enable specific targeting and cause very limited side effects of such an intervention. This is particularly true if its specificity and the ratio between beneficial and detrimental effects of treatment with anti-TUCAP1 antibody would be compared to similar approaches involving blocking antibodies in cancer such as anti-TNF or anti-growth factor receptors.

The examples above disclose particular embodiments of the invention in detail. However, this has been done by way of example and for the purposes of illustration only. The examples are not intended to limit the scope of the appended claims, which define the invention.

Sequences table:

| Sequence number | Description |
|---|---|
| SEQ ID NO 1 | TM9SF4: Encoding sequence for the full protein |
| SEQ ID NO 2 | TM9SF4: Amino acid sequence for the full protein |
| SEQ ID NO 3 | TM9SF2: Encoding sequence for the full protein |
| SEQ ID NO 4 | TM9SF2: Amino acid sequence for the full protein |
| SEQ ID NO 5 | TM9SF3: Encoding sequence for the full protein |
| SEQ ID NO 6 | TM9SF3: Amino acid sequence for the full protein |
| SEQ ID NO 7 | TM9SF1: Encoding sequence for the full protein |
| SEQ ID NO 8 | TM9SF1: Amino acid sequence for the full protein |
| SEQ ID NO 9 | TM9SF4: Primer for TM9SF4 detection - forward |
| SEQ ID NO 10 | TM9SF4: Primer for TM9SF4 detection - reverse |
| SEQ ID NO 11 | TM9SF4: Primer for His-tagged N-terminal domain |
| | synthesis of TUCAP1 - forward |
| SEQ ID NO 12 | TM9SF4: Primer for His-tagged N-terminal domain |
| | synthesis of TUCAP1 - reverse |
| SEQ ID NO 13 | GAPDH: Primer for GAPDH detection - forward |
| SEQ ID NO 14 | GAPDH: Primer for GAPDH detection - reverse |
| SEQ ID NO 15 | TM9SF4: Primers used to direct GFP-tagged full |
| | length TM9SF4 - forward |
| SEQ ID NO 16 | TM9SF4: Primers used to direct GFP-tagged full |
| | length TM9SF4 - reverse |
| SEQ ID NO 17 | TM9SF4: Amino acid sequence for His tagged |
| | TM9SF4 aa 18-279 |
| SEQ ID NO 18 | TM9SF4: Amino acid sequence corresponding to |
| | TM9SF4 aa 221-235 |
| SEQ ID NO 19 | TM9SF1: Amino acid sequence for His tagged |
| | TM9SF1 aa 90-215 |
| SEQ ID NO 20 | TM9SF2: Amino acid sequence for His tagged |
| | TM9SF2 aa 106-271 |
| SEQ ID NO 21 | TM9SF3: Amino acidic sequence for His tagged |
| | TM9SF3 aa 29-222 |
| SEQ ID NO 22 | TM9SF4: Nucleotides sequence 287-1072 of human |
| | TM9SF4 (NM_014742.3 GI: 164519075) |
| SEQ ID NO 23 | TM9SF4: Oligonucleotidic forward primer to amplify |
| | SEQ ID NO 22 from cDNA |
| SEQ ID NO 24 | TM9SF4: Oligonucleotidic reverse primer to amplify |
| | SEQ ID NO 22 from cDNA |
| SEQ ID NO 25 | TM9SF4: Forward primer with LIC-extension for |
| | cloning steps |
| SEQ ID NO 26 | TM9SF4: Reverse primer with LIC-extension for |
| | cloning steps |
| SEQ ID NO 27 | p2N N-terminal His Tag amino acid sequence |
| SEQ ID NO 28 | TM9SF1: Nucleotides sequence 616-993 of human |
| | TM9SF1(NM_006405.5; GI: 62460631 |
| | variant 1; NM_001014842.1; GI: 62460634 variant 2) |
| SEQ ID NO 29 | TM9SF1: Oligonucleotidic forward primer to amplify |
| | SEQ ID NO 28 from cDNA |
| SEQ ID NO 30 | TM9SF1: Oligonucleotidic reverse primer to amplify |
| | SEQ ID NO 28 from cDNA |
| SEQ ID NO 31 | TM9SF2: Nucleotides sequence 449-946 of human |
| | TM9SF2 (NM_004800.1 GI: 4758873) |
| SEQ ID NO 32 | TM9SF2: Oligonucleotidic forward primer to amplify |
| | SEQ ID NO 31 from cDNA |
| SEQ ID NO 33 | TM9SF2: Oligonucleotidic reverse primer to amplify |
| | SEQ ID NO 31 from cDNA |
| SEQ ID NO 34 | TM9SF3: Nucleotides sequence 302-883 of human |
| | TM9SF3 (NM_020123.3 GI: 190194385) |
| SEQ ID NO 35 | TM9SF3: Oligonucleotidic forward primer to amplify |
| | SEQ ID NO 34 from cDNA |
| SEQ ID NO 36 | TM9SF3: Oligonucleotidic reverse primer to amplify |
| | SEQ ID NO 34 from cDNA |
| SEQ ID NO 37 | Invitrogen siRNA oligo for TM9SF4 silencing |
| SEQ ID NO 38 | Invitrogen siRNA oligo for TM9SF4 silencing |

### REFERENCES

1. Zaravinos A, Lambrou G I, Boulalas I, Delakas D, Spandidos D A. Identification of common differentially expressed genes in urinary bladder cancer. PLoS One. 2011Apr. 4; 6(4):e18135.
2. He P, Peng Z, Luo Y, Wang L, Yu P, Deng W, An Y, Shi T, Ma D. High-throughput functional screening for autophagy-related genes and identification of TM9SF1 as an autophagosome-inducing gene. Autophagy. 2009 January; 5(1):52-60.
3. Chang H, Jeung H C, Jung J J, Kim T S, Rha S Y, Chung H C. Identification of genes associated with chemosensitivity to SAHA/taxane combination treatment intaxane-resistant breast cancer cells. Breast Cancer Res Treat. 2011 January; 125(1):55-63.
4. Mackinnon R N, Selan C, Wall M, Baker E, Nandurkar H, Campbell L J. The paradox of 20q11.21 amplification in a subset of cases of myeloid malignancy with chromosome 20 deletion. Genes Chromosomes Cancer. 2010 November; 49(11):998-1013.
5. Lozupone F, Perdicchio M, Brambilla D, Borghi M, Meschini S, Barca S, Marino M L, Logozzi M, Federici C, Iessi E, de Milito A, Fais S. The human homologue of Dictyostelium discoideum phg1A is expressed by human metastatic melanoma cells. EMBO Rep. 2009 December; 10(12):1348-54.
6. Fais S. Cannibalism: A way to feed on metastatic tumors. (2007) Cancer Lett; 258: 155-164.
7. Breier F, Feldmann R, Fellenz C, Neuhold N, Gschnait F. Primary invasive signet-ring cell melanoma. (1999) J Cutan Pathol 10:533-536.
8. Caruso R A, Muda A O, Bersiga A, Rigoli L, Inferrera C. Morphological evidence of neutrophil tumor cell phagocytosis (cannibalism) in human gastric adenocarcinomas. (2002) Ultrastruct Pathol 26:315-321.
9. DeSimone P A, East R, Powell R D. Phagocytic tumor cell activity in oat cell carcinoma of the lung. (1980) Hum Pathol; 11:535-539.
10. Kojima S, Sekine H, Fukui I, Ohshima H. Clinical significance of “cannibalism” in urinary cytology of bladder cancer. (1998) Acta Cytol 42:1365-1369.
11. Lugini L, Matarrese P, Tinari A, Lozupone F, Federici C, Iessi E, Gentile M, Luciani F, Parmiani G, Rivoltini L, Malorni W, Fais S. Cannibalism of live lymphocytes by human me tastatic but not primary melanoma cells (2006) Cancer Res. 66:3629-3638.
12. Santoni G, Farfariello V. TRP channels and cancer: new targets for diagnosis and chemotherapy. Endocr Metab Immune Disord Drug Targets. 2011 Mar. 1; 11(1):54-67.
13. Shimizu T, Lee E L, Ise T, Okada Y. Volume-sensitive Cl(−) channel as a regulator of acquired cisplatin resistance. Anticancer Res. 2008 January-February; 28(1A):75-83
14. Huang Y, Sadée W. Membrane transporters and channels in chemoresistance and -sensitivity of tumor cells. Cancer Lett. 2006 Aug. 8; 239(2):168-82. Epub 2005 Oct. 1: Huber V, De Milito A, Harguindey S, Reshkin S J, Wahl M L, Rauch C, Chiesi A, Pouysségur J, Gatenby R A, Rivoltini L, Fais S. Proton dynamics in cancer. J Transl Med. 2010 Jun. 15; 8:57.
15. De Milito A, Fais S. Tumor acidity, chemoresistance and proton pump inhibitors. Future Oncol. 2005 December; 1(6):779-86.
16. Sloane B F Yan S, Podgorski I, Linebaugh B E, Cher. M L, Mai J, Cavallo-Medved D, Sameni M, Dosescu J, Moin K. Cathepsin B and tumor proteolysis: contribution of the tumor microenvironment. Seminars in Cancer Biology 15 (2005) 149-157.
17. Gatenby R A, Gawlinski E T. The glycolytic phenotype in carcinogenesis and tumor invasion: insights through mathematical models. Cancer Res. 2003, 63(14):3847-54.
18. Thery C, Zitvogel L and Amigorena S Exosomes: composition, biogenesis and function. (2002) Nat Rev Immunol 2: 569-579
19. Rak J. Microparticles in cancer. Semin Thromb Hemost. 2010 November; 36(8):888-906.
20. Logozzi M, De Milito A, Lugini L, Borghi M, Calabrò L, Spada M, Perdicchio M, Marino M L, Federici C, lessi E, Brambilla D, Venturi G, Lozupone F, Santinami M, Huber V, Maio M, Rivoltini L, Fais S. High levels of exosomes expressing CD63 and caveolin-1 in plasma of melanoma patients. PLoS One. 2009; 4(4):e5219.
21. Iero M, Valenti R, Huber V, Filipazzi P, Parmiani G, Fais S, Rivoltini L. Tumour-released exosomes and their implications in cancer immunity. Cell Death Differ. 2008, 15(1):80-8.

What is claimed is:

1. A hybridoma cell line selected from the group consisting of hybridoma cell lines 1A4-A3, 1A4-A8, 1A4-F2, 1A4-G1, 5C1-B4, 5C1-C5, 5C1-D4, and 5C1-G6.

2. An isolated monoclonal antibody or a fragment thereof, wherein the antibody is produced by the hybridoma cell line of claim 1.

3. The monoclonal antibody or the fragment of claim 2, wherein the antibody or the fragment is conjugated to a detectable label.

4. The monoclonal antibody or the fragment of claim 2, wherein the antibody or the fragment is conjugated to a drug.

5. The monoclonal antibody of claim 4, wherein the drug is a medicinal compound approved for cancer therapy.

6. The monoclonal antibody of claim 4, wherein the drug is a medicinal compound approved for treatment of a TM9SF4 protein related disease condition.

7. An isolated mouse anti-TMSF4 antibody or an antigen binding fragment thereof, wherein the antibody binds to SEQ ID NO: 17 of TMSF4-protein and wherein the antibody is produced by a hybridoma cell line of claim 1.

8. An isolated mouse anti-TMSF1 antibody or antigen binding fragment thereof, wherein the antibody binds to SEQ ID NO: 19 of TMSF1-protein.

9. An isolated mouse anti-TMSF-2 antibody or antigen binding fragment thereof, wherein the antibody binds to SEQ ID NO: 20 of TMSF2-protein.

10. An isolated mouse anti-TMSF-3 antibody or antigen binding fragment thereof, wherein the antibody binds to SEQ ID NO: 21 of TMSF3-protein.

11. A kit to detect TM9SF4-protein, said kit comprising clones of monoclonal antibodies or fragments thereof of claim 2 and a microplate or microtiter plate.

12. The kit of claim 11, wherein the kit further comprises purified TM9SF4 N-terminal domain fragment corresponding to SEQ ID NO:17 and optionally isolated TM9SF4 protein according to SEQ ID NO:2, rabbit anti-TM9SF4 polyclonal antibodies, a biotinylated anti-mouse IgG, horseradish peroxidase (HRP)-conjugated streptavidin, a HRP-conjugated anti mouse secondary antibody, a biotinylated anti-rabbit IgG or a HRP-conjugated anti rabbit secondary antibody, a phosphate buffer solution, a washing buffer solution, a diluent for a biological sample suspected to contain TM9SF4, a chromogenic or luminometric substrate, and a stop solution.

13. The kit of claim 11, wherein the kit is for an Enzyme-Linked Immunosorbent Assay (ELISA) and wherein the monoclonal antibodies are bound to the microplate or the microtiter plate.

14. A pharmaceutical composition comprising therapeutically effective amount of an antibody of claim or an antigen binding fragment thereof.
